(19) United States
(12) Patent Application Publication
(10) Pub. No.: US 2026/0265242 A1
(43) Pub. Date: Sep. 10, 2026

(54) FABK INHIBITOR COMPOSITIONS

(71) Applicants: The Texas A&M University System, College Station, TX (US); University of Tennessee Research Foundation, Memphis, TN (US); University of Hawai'i, Honolulu, HI (US)

(72) Inventors: Kirk Hevener, Memphis, TN (US); Dianqing Sun, Honolulu, HI (US); Julian G. Hurdle, College Station, TX (US)

(73) Assignees: The Texas A&M University System, College Station, TX (US); University of Tennessee Research Foundation, Memphis, TN (US); University of Hawai'i, Honolulu, HI (US)

(21) Appl. No.: 19/166,422

(22) PCT Filed: Mar. 17, 2024
(86) PCT No.: PCT/US2024/020337

§ 371 Date: Sep. 17, 2025

Related U.S. Application Data
(60) Provisional application No. 63/490,897, filed on Mar. 17, 2023.

(51) Int. Cl. C07D 417/14; A61K 31/4178; A61K 31/4184; A61K 31/423; A61K 31/427; A61K 31/428; A61K 31/437; A61K 31/4439; A61K 31/454; A61K 31/4545; A61K 31/4709; A61K 31/496; A61K 31/497; A61K 31/501; A61K 31/502; A61K 31/506; A61K 31/513; A61K 31/517; A61K 31/52; A61K 31/675; C07D 235/30; C07D 263/58; C07D 277/82; C07D 403/12; C07D 417/12; C07D 473/38; C07D 498/04; C07F 9/6558

(52) U.S. Cl. CPC C07D 417/14; A61K 31/4178; A61K 31/4184; A61K 31/423; A61K 31/427; A61K 31/428; A61K 31/437; A61K 31/4439; A61K 31/454; A61K 31/4545; A61K 31/4709; A61K 31/496; A61K 31/497; A61K 31/501; A61K 31/502; A61K 31/506; A61K 31/513; A61K 31/517; A61K 31/52; A61K 31/675; C07D 235/30; C07D 263/58; C07D 277/82; C07D 403/12; C07D 417/12; C07D 473/38; C07D 498/04; C07F 9/65583

(57) ABSTRACT

Embodiments of the invention are directed to inhibitors of FabK activity. Embodiments of the inventions are directed to compounds having a structure selected from the group consisting of an imidazole scaffold, a thiazole scaffold, a pyridine-thiazole scaffold, a benzothiazole scaffold, a benzimidazole scaffold, a benzoxazole scaffold and combinations thereof.

### CROSS-REFERENCE TO RELATED APPLICATION(S)

This application claims priority to U.S. Provisional Patent Application No. 63/490,897 filed Mar. 17, 2023, the entire specification of which is fully incorporated by reference.

### STATEMENT REGARDING FEDERALLY SPONSORED RESEARCH

This invention was made with government support under federal grant number W81XWH-20-1-0296 awarded by the Department of Defense and federal grant number R21AI126755 awarded by the National Institutes of Health. The government has certain rights in the invention.

### TECHNICAL FIELD

The invention relates generally to FabK (Fatty Acid Synthesis Protein Enovl-ACP Reductase II) inhibitor compositions having antimicrobial activity. Specifically, embodiments of the invention are directed to FabK inhibitors and their use in the treatment of infections caused by microbes such as C. difficile, F. nucleatum and P. gingivalis.

### BACKGROUND

Clostridioides difficile infection (CDI) is a leading cause of healthcare-associated diarrhea that is often associated with the use of broad-spectrum antibacterial agents.

According to the CDC, C. difficile caused approximately 223,900 cases in hospitalized patients and 12,800 deaths in 2017 in the United States. Prior to the approval of fidaxomicin in 2011, metronidazole and vancomycin were the two main antibiotic treatments for CDI. However, metronidazole is not recommended as a first-line CDI medication in the 2021 IDSA/SHEA and ESCMID CDI guidelines, with fidaxomicin recommended as the first-line treatment and vancomycin as its alternate. Fidaxomicin is a narrow-spectrum anti-C. difficile agent with limited activity against other gut bacteria compared to vancomycin, which has a broader spectrum of activity. However, resistance to fidaxomicin has emerged and alternative CDI antimicrobials are needed for recurrent CDI to complement current non-antimicrobial treatments for recurrent CDI, i.e., fecal microbiota transplantation (FMT) and adjunctive treatment with bezlotoxumab, a monoclonal antibody. Therefore, inhibitors of enoyl-acyl carrier protein (enoyl-ACP) reductase FabK, which is involved in de novo fatty acid synthesis, may present good options.

The bacterial type II fatty acid synthesis pathway (FAS-II) comprises a series of individual enzymatic steps to synthesize critical lipids required for bacterial membranes. Lipophilic substrates of the FAS-II enzymes are shuttled between the enzyme steps by an acyl-linked carrier protein (ACP). Several FAS-II steps are conducted by species-specific isozymes that may be exploited as narrow-spectrum antibacterial targets. The rate-limiting enoyl-ACP reductase step, responsible for the reduction of a double bond, has four known major isozymes—FabI, FabL, FabV, and FabK. The FabK isozyme is structurally and mechanistically distinct from the other enoyl-ACP reductase isozymes and presents an opportunity for the design of agents targeting organisms that solely express this enzyme. C. difficile is one such organism. C. difficile FabK (CdFabK) is essential for growth even in the presence of host fatty acids.

Other pathogens carrying the FabK enzyme also include Fusobacterium species (for example, F. nucleatum), Porphyromonas species (for example, P. gingivalis) and Streptococcus species (for example, S. mutans). F. nucleatum is implicated in multiple diseases including periodontal disease, pre-term births, inflammatory bowel disease, and colorectal (CRC) and breast cancers. In periodontal disease, F. nucleatum assists periodontal pathogens such as Porphyromonas gingivalis by promoting their integration into the oral microbiota community. In the context of CRC, genomic analysis of patient biopsies has shown a frequent association of F. nucleatum with tumor tissues, as opposed to normal tissues. F. nucleatum expresses virulence factors that contribute to the development of a pro-tumorigenic environment, which includes suppression of the immune system, promotion of chemoresistance, and induction of inflammation. Similar to C. difficile, F. nucleatum and P. gingivalis depend on the FAS-II pathway to synthesize β-Hydroxyacyl-ACPs, which are essential precursors for the lipid component of lipopolysaccharide outer membranes. Therefore, targeting FabK, which is a required enzyme in the FAS-II pathway, provides an avenue for the development of narrow-spectrum antimicrobials, while minimizing collateral damage of the normal microbiota in the human body.

The pathogens discussed above are either associated with, or present risk factors for, a range of diseases, ranging from diseases of the oral cavity (gum diseases—for example, periodontal disease and gingivitis-; cariogenic diseases—for example, dental caries; halitosis); gastrointestinal and systemic diseases (for example, colorectal cancer, cardiovascular disease, atherosclerosis, cerebral aneurysm, inflammatory bowel disease, appendicitis, Lemierre's syndrome, rheumatoid arthritis, and pre-term births, and Alzheimer's). As such, inhibitors of FabK could have broad applicability in treating microbial infections.

### SUMMARY OF THE INVENTION

An embodiment of the invention is directed to a composition comprising an inhibitor of FabK activity. In certain embodiments, the claimed inhibitor is a compound having a structure selected from the group consisting of an imidazole scaffold, a thiazole scaffold, a pyridine-thiazole scaffold, a benzothiazole scaffold, a benzimidazole scaffold, a benzoxazole scaffold and combinations thereof. In certain embodiments, the inhibitor is a unsubsituted or substituted phenylimidazole compound

### BRIEF DESCRIPTION OF THE DRAWINGS

FIG. 1 shows potential structural modifications of phenylimidazole analogs.

FIG. 2 shows the synthesis of compounds 5a-5i; a) 10, K₂CO₃, DMF. 80° C., 1.5 h. 25-91%; b) CDI, DCM, rt; c) MeONa, MeOH, rt, o/n, then NH₄Cl, rt, o/n, 86%; d) 15, K₂CO₃, 80° C., o/n, 37%; e) HCl, H₂O, 100° C., 91%; f) Et₃N, DCM, rt, 80 min, 30-99%.

FIG. 3 shows the synthesis of compounds 6a-6l; a) CDI, DCM, rt, o/n; b) 16, Et₃N, DCM, rt, o/n, 13-98% over two steps.

FIG. 4 shows the synthesis of compounds 8a-8c; a) CDI, DCM, rt, o/n; b) Et₃N, DCM, rt, o/n, 53-72%.

FIG. 5 shows the general structure of a compound having a thiazole scaffold.

FIG. 6 shows the general structure of a compound having a benzothiazole/benzimidazole/benzoxazole scaffold.

FIG. 7 is a listing of representative compounds of the claimed invention.

### DETAILED DESCRIPTION

An embodiment of the claimed invention is directed to a composition comprising an antimicrobial agent. In certain embodiments, the antimicrobial agent comprises an inhibitor of FabK enzyme.

In other embodiments, the antimicrobial agent is a compound having a scaffold selected from the group consisting of an imidazole scaffold, a thiazole scaffold, a pyridine-thiazole scaffold, a benzothiazole scaffold, a benzimidazole scaffold, a benzoxazole scaffold and combinations thereof.

In certain embodiments, the antimicrobial agent is a phenylimidazole compound. In some embodiments, the phenylimidazole compound contains substitutions. In certain embodiments, the anti-C. difficile agent is a phenylimidazole compound having a substitution to the pyridine head group. In other embodiments, the phenylimidazole compound has a modification to the pyridine head group. In certain embodiments, the head group is modified by the addition of halogen substitutions.

In certain embodiments, the phenylimidazole compound comprises modifications to the planar central aromatic region. In certain embodiments, the thiazole of the central aromatic region is substituted with quinoline and/or benzothiazole. In other embodiments, the urea is replaced by a thiourea.

In certain embodiments, the phenylimidazole compound comprises modifications to the aromatic tail group ring system involving substitution of the phenylimidazole group with varying aromatic bicyclic systems.

Embodiments of the invention are directed to the use of the claimed compounds as inhibitors of microbial infections in which the microbes contain FabK. In certain embodiments that claimed compounds are used as the sole inhibitor of FabK. In other embodiments, the claimed compounds are used in conjunction with other antimicrobial agents.

In certain embodiments, the claimed compounds may be used to treat inflammatory diseases in which the progenitor is a microbial pathogen carrying FabK enzyme.

In certain embodiments, the claimed compounds may be formulated for administration in compositions that are either dispersed in formulation or attached, for example, to polymeric materials designed to be embedded in a human or animal subject. The compounds may be delivered via different routes of administration, including topical, oral administration, inhalation, trans or sub-dermal, and rectal.

In certain embodiments, compositions comprising one or more of the claimed compounds may be used to modulate or alter the composition of the human microbiome (microbiota) to produce beneficial health effects, for example to increase microbial community associated with producing secondary bile that protects against infection, or modulate microbiome metabolism in a way to produce other metabolites that are beneficial to human health.

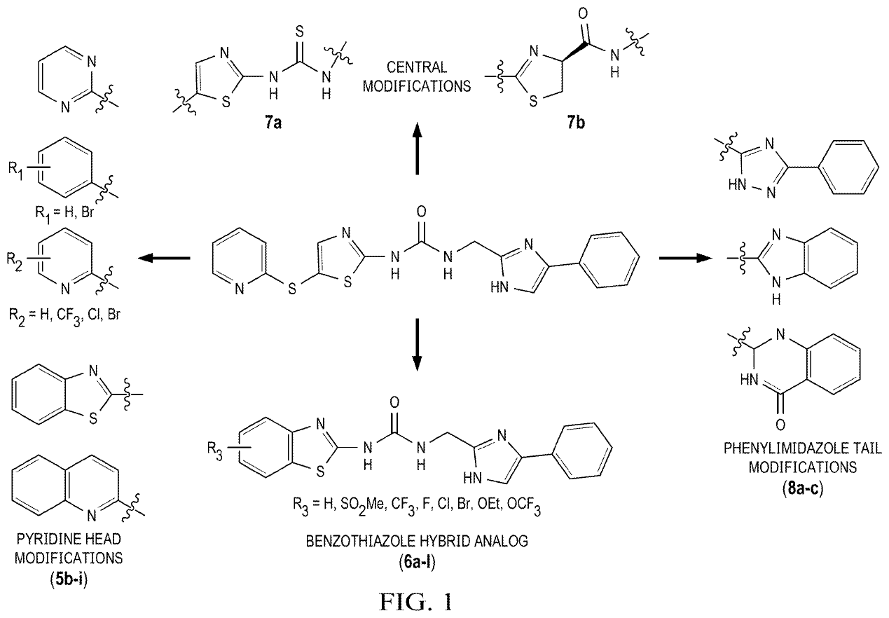

FIG. 1 shows the potential structural modifications of phenylimidazole analogs that form a part of the claimed invention. The reference labels 5b-i. 6a-l, 7a, 7b and 8a-c refer to representative compounds that were synthesized and/or used as starting compounds to synthesize the claimed compounds.

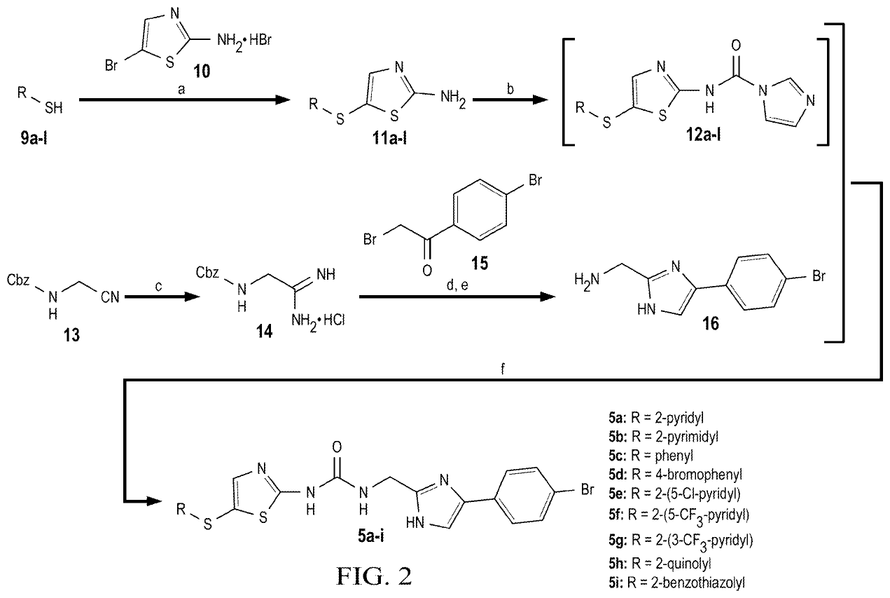

FIG. 2 shows a schematic for the synthesis of compounds 5a-5i. The reference labels 5a-i, 5a, 5b, 5c, 5d, 5e, 5f, 5g, 5 h, 5i, 9a-l, 11a-l, 12a-l, 13, 14 and 16 refer to representative compounds that were synthesized and/or used as starting compounds to synthesize the claimed compounds.

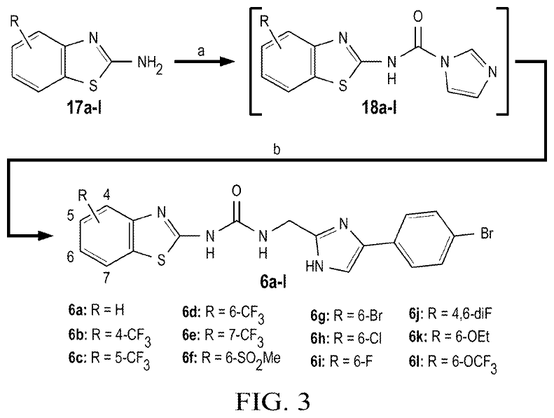

FIG. 3 shows a schematic for the synthesis of compounds 6a-6l. The reference labels 17a-l, 18a-l, 6a-l, 6a, 6b, 6c, 6d, 6e, 6f, 6g, 6h, 6i, 6j, 6k and 6l refer to representative compounds that were synthesized and/or used as starting compounds to synthesize the claimed compounds.

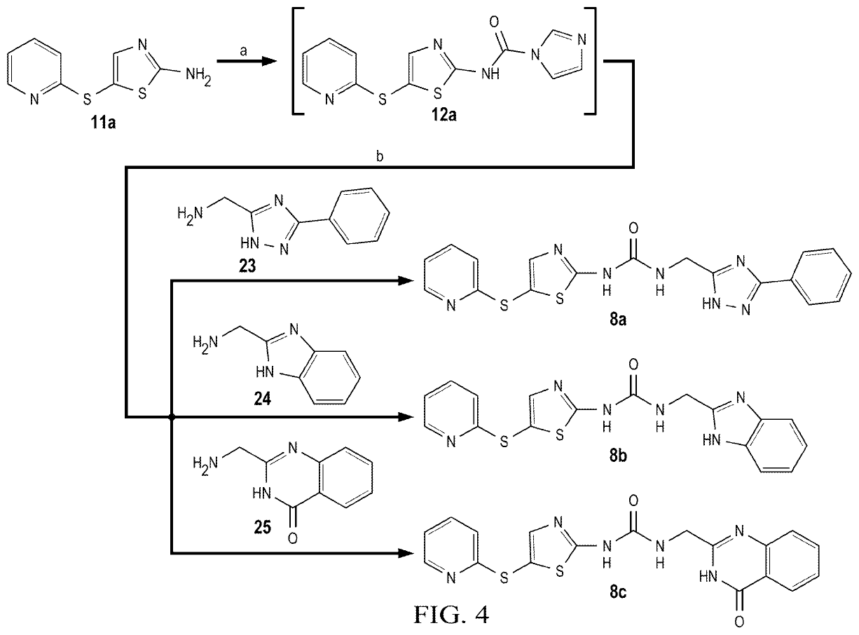

FIG. 4 shows a schematic for the synthesis of compounds 8a-8c. The reference labels 11a, 12a, 23, 24, 25, 8a, 8b and 8c refer to representative compounds that were synthesized and/or used as starting compounds to synthesize the claimed compounds.

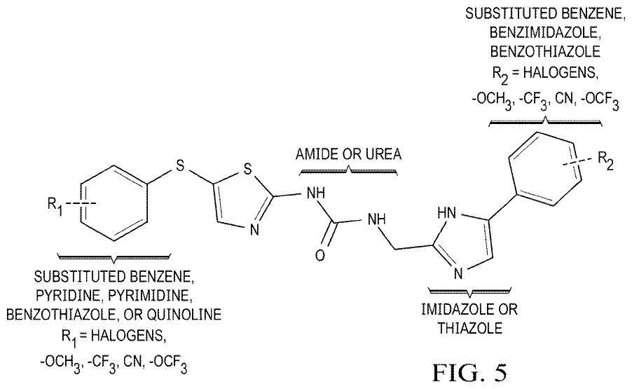

FIG. 5 shows a structure of a compound having a thiazole scaffold. The ring compounds are selected from benzene, pyridine, pyrimidine, benzothiazole, benzimidazole or quinoline. R₁ and R₂ is a halogen, methoxy, trifluoro-methyl, cyano or trifluoro-methoxy functional group.

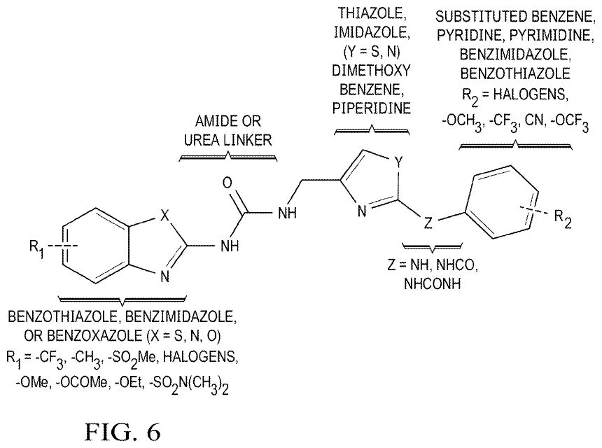

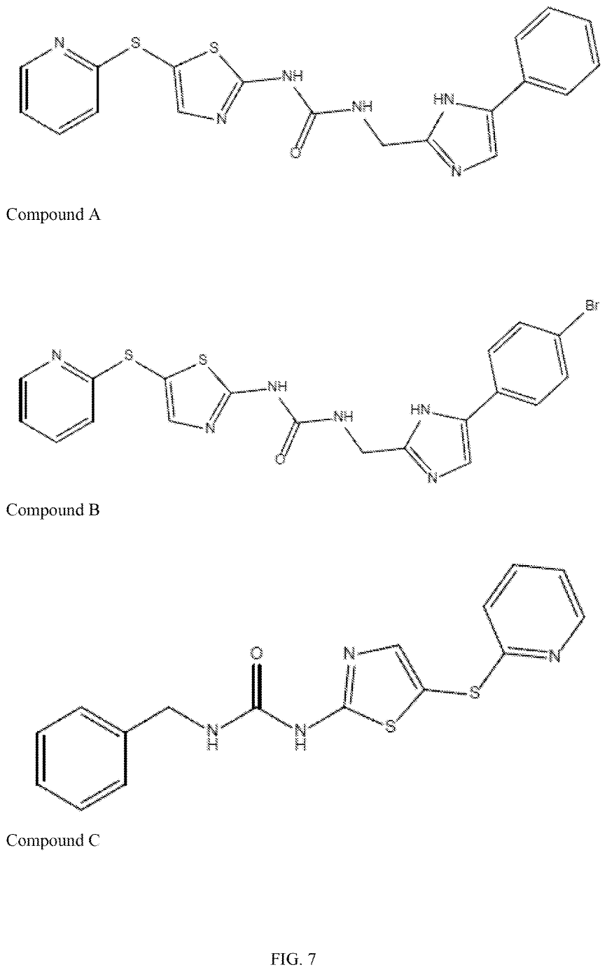

FIG. 6 shows a structure of a compound having a benzothiazole/benzimidazole/benzoxazole scaffold. The ring compounds are selected from benzene, pyridine, pyrimidine, benzothiazole or benzimidazole. R₁ and R₂ is a halogen, trifluoromethyl, methyl, methoxy, mesylate, methoxy, methyl-carbonyl, ethoxy, or sulfonamide functional group.

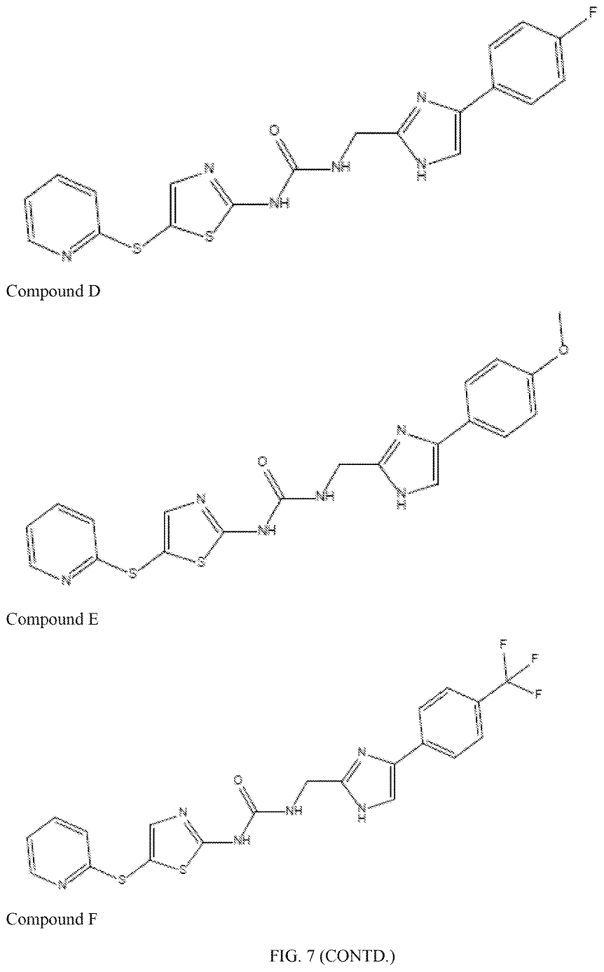

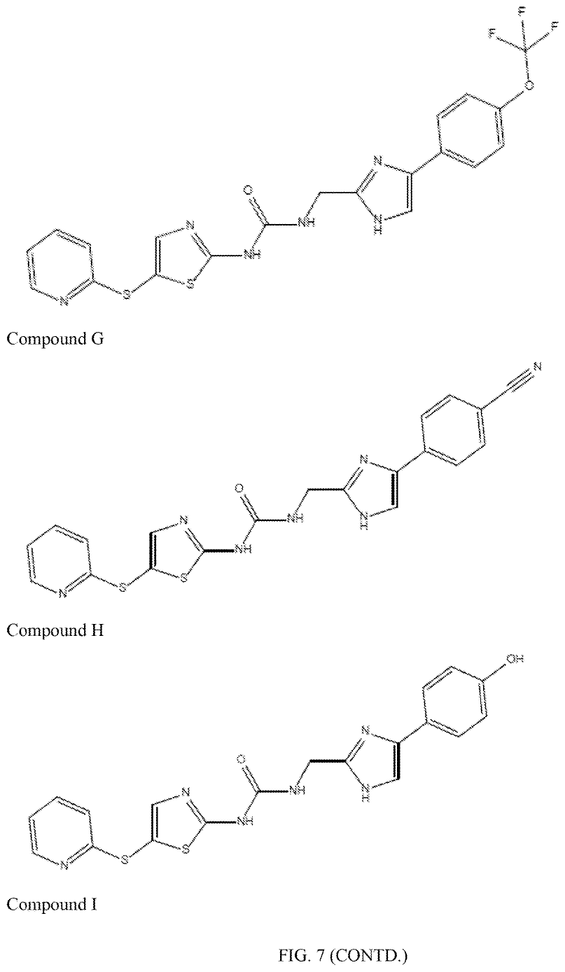

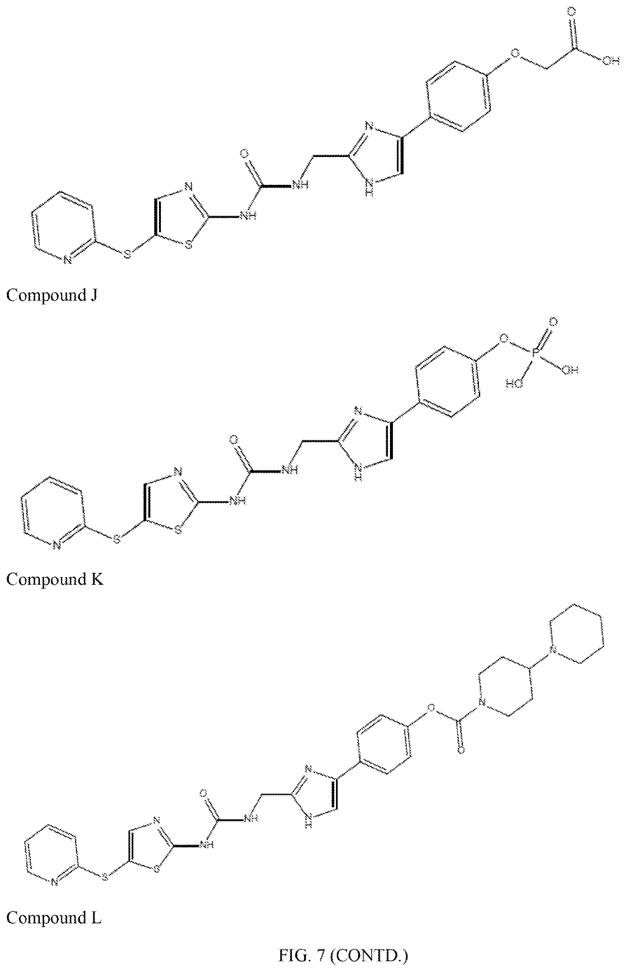

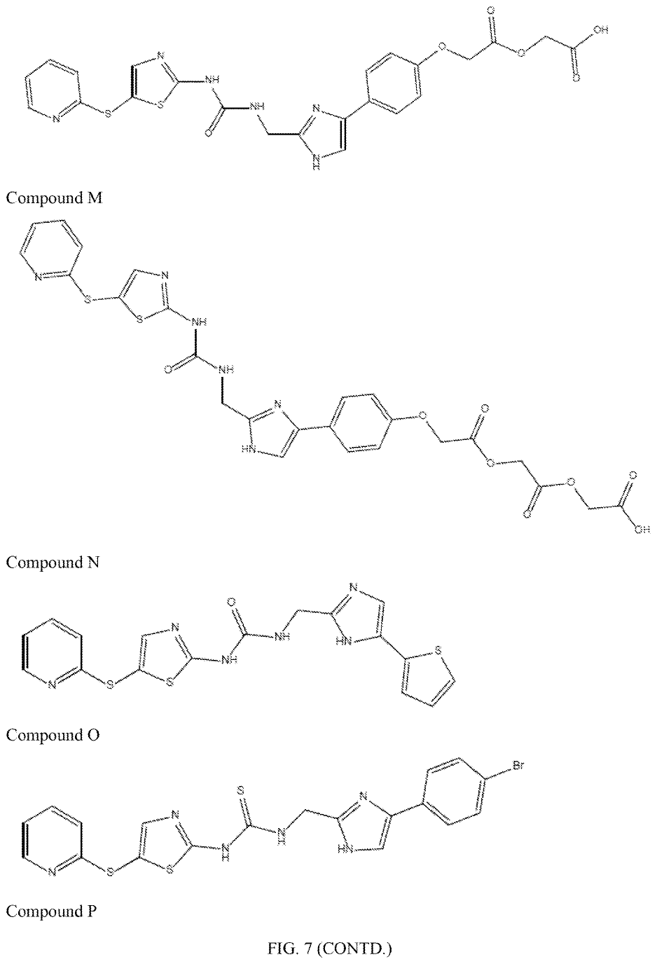

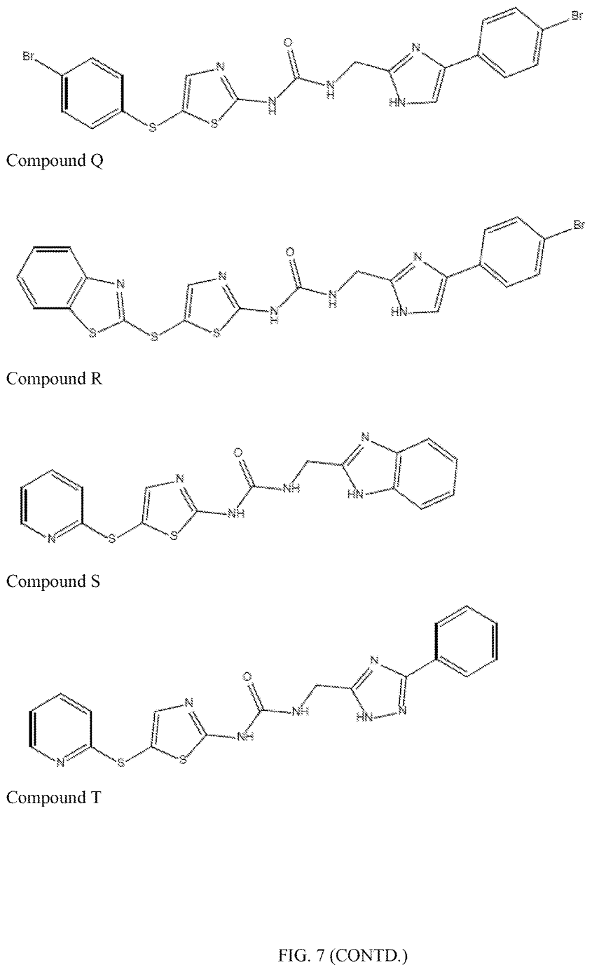

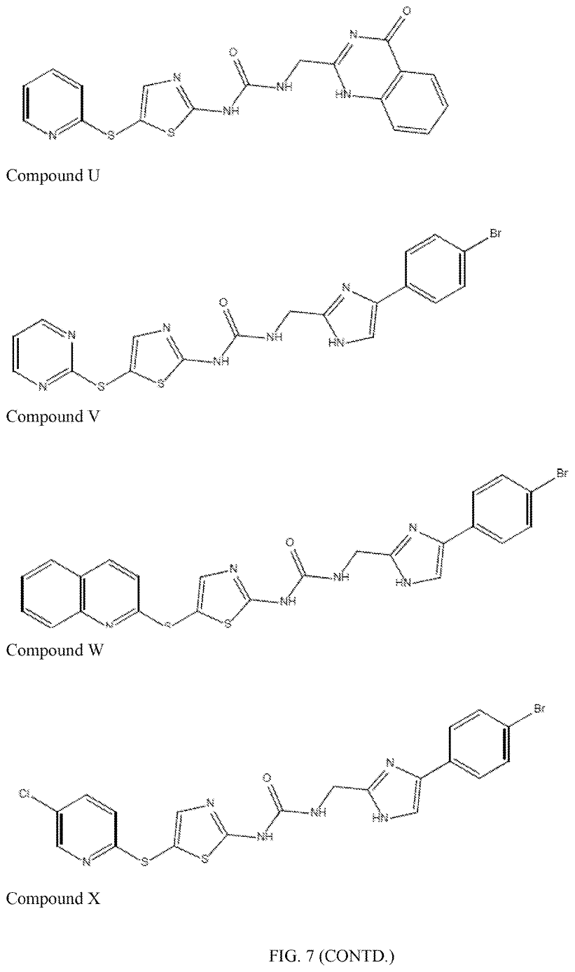

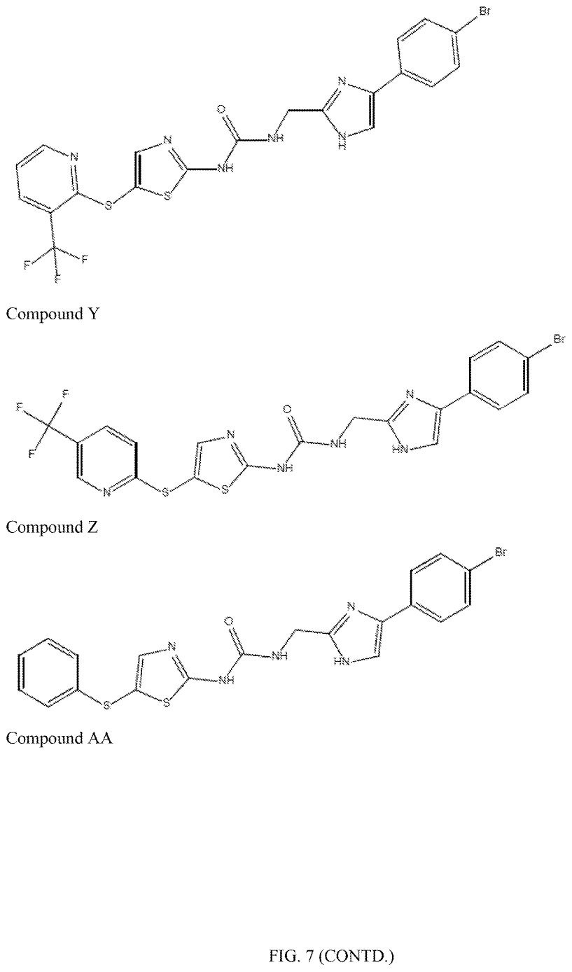

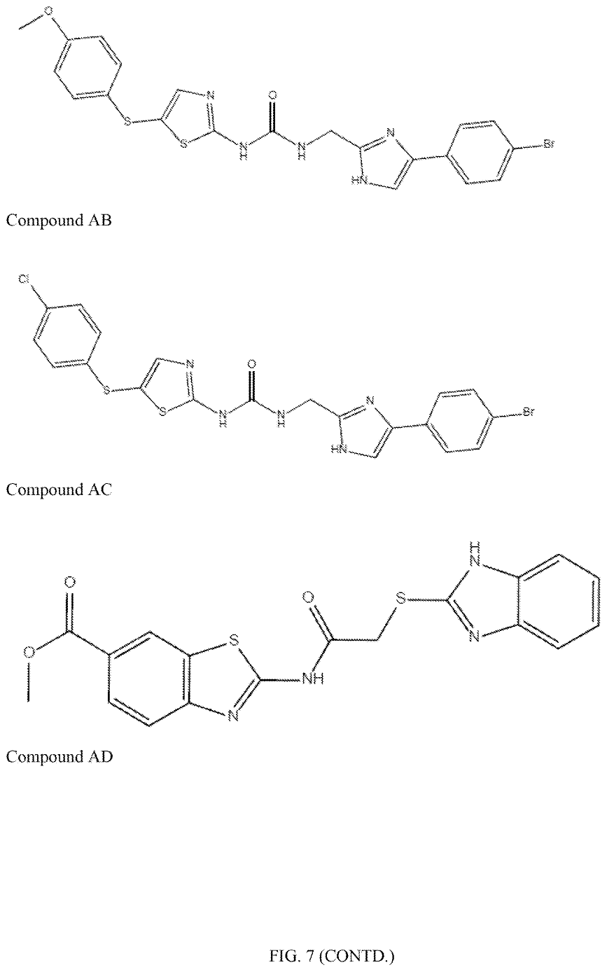

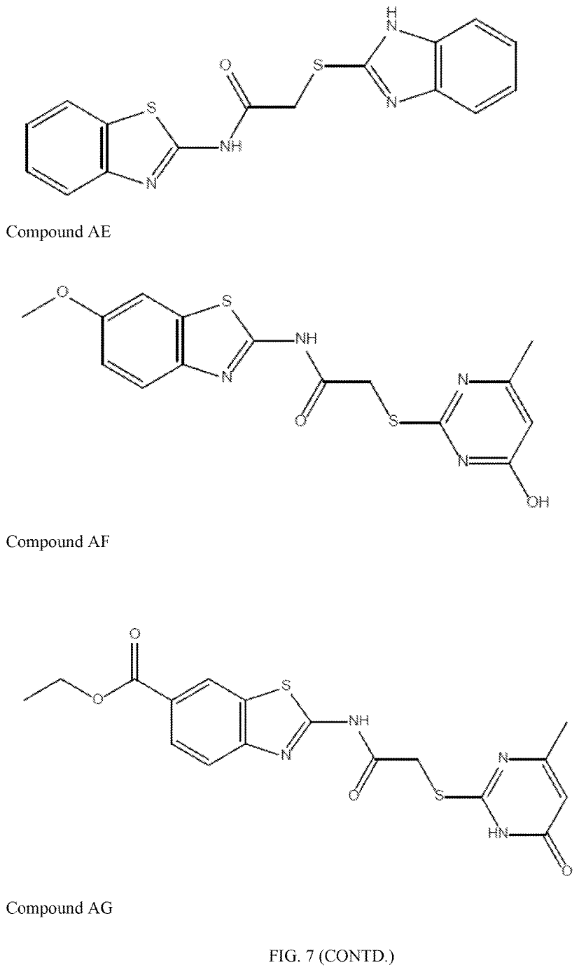

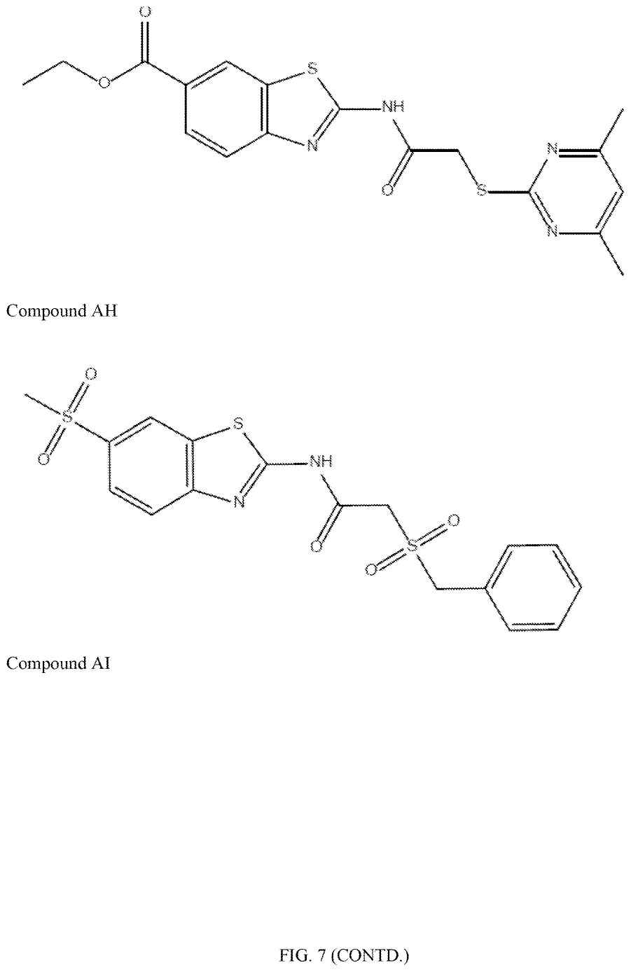

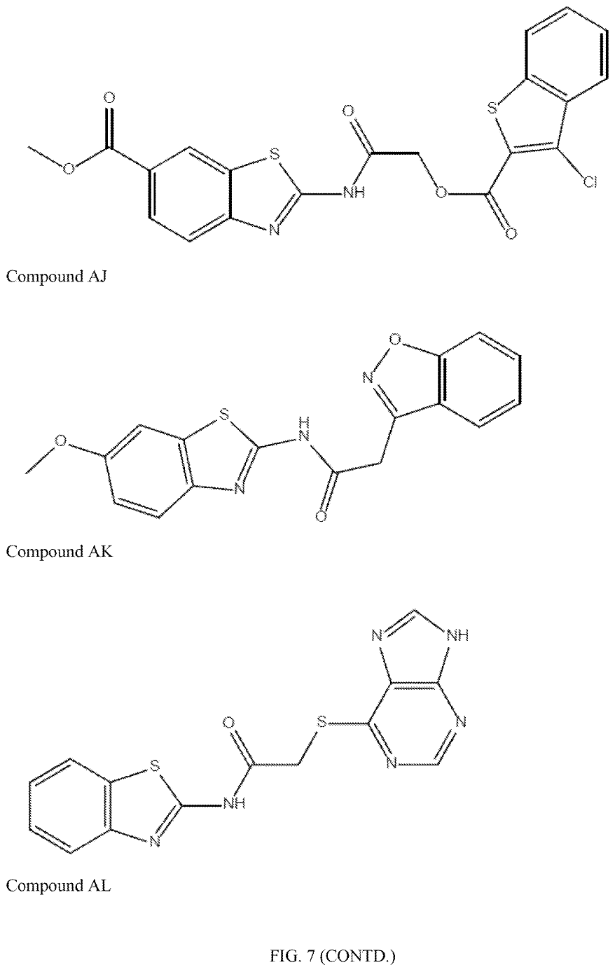

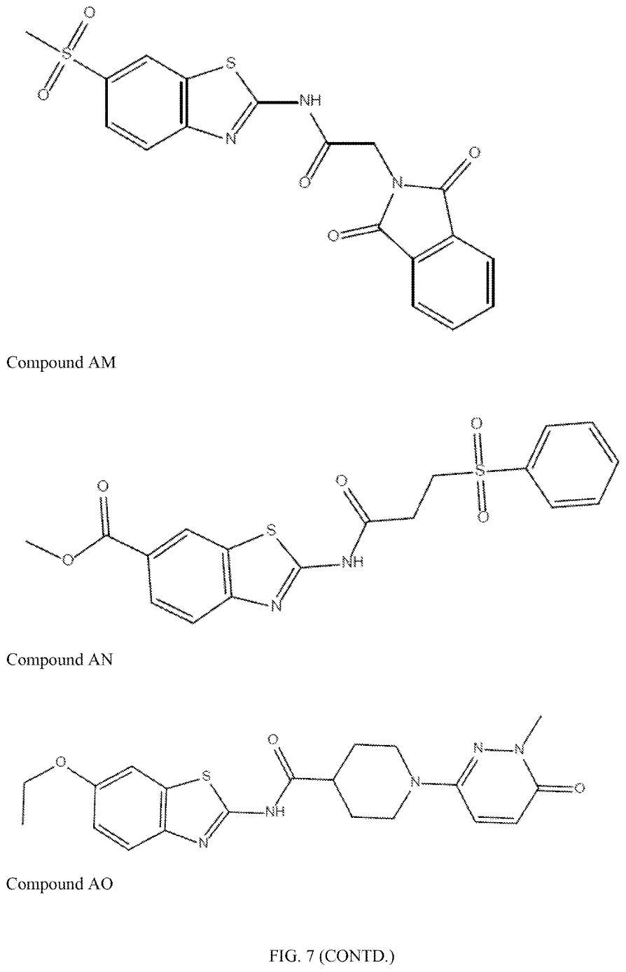

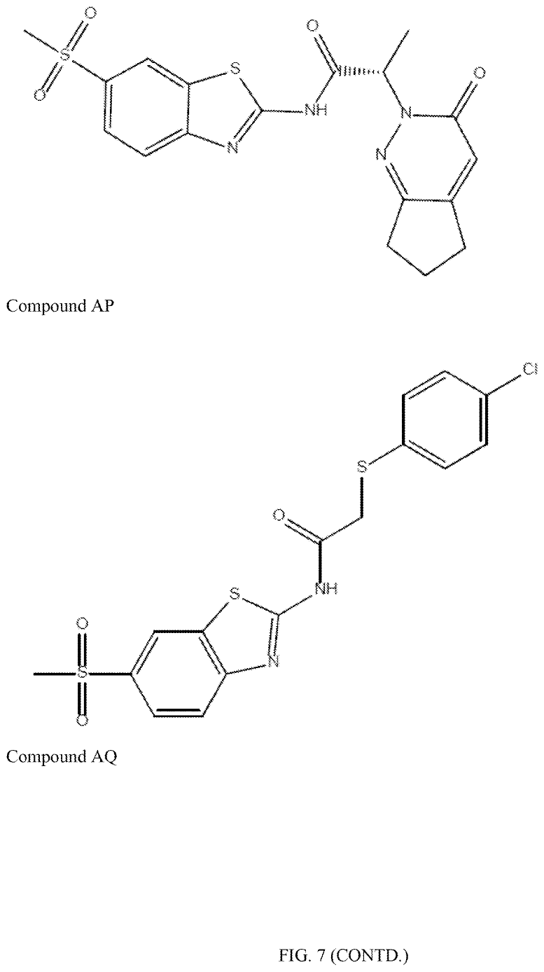

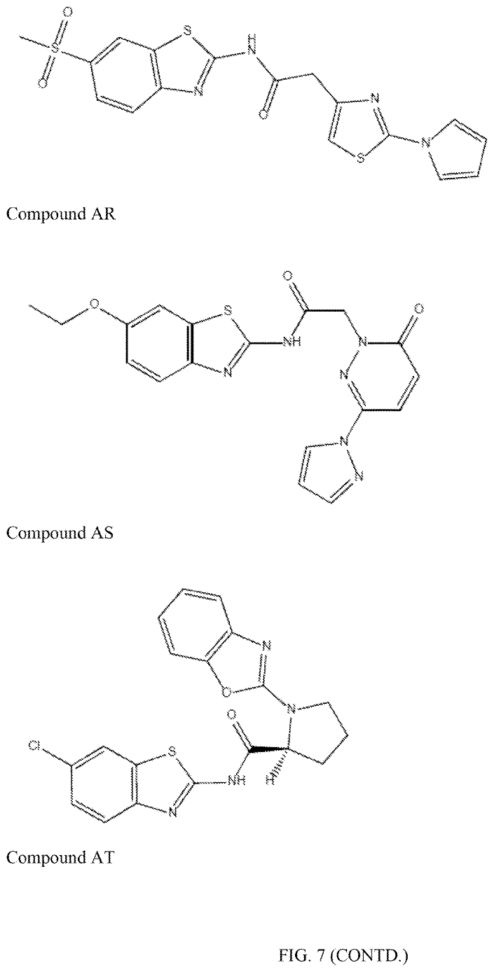

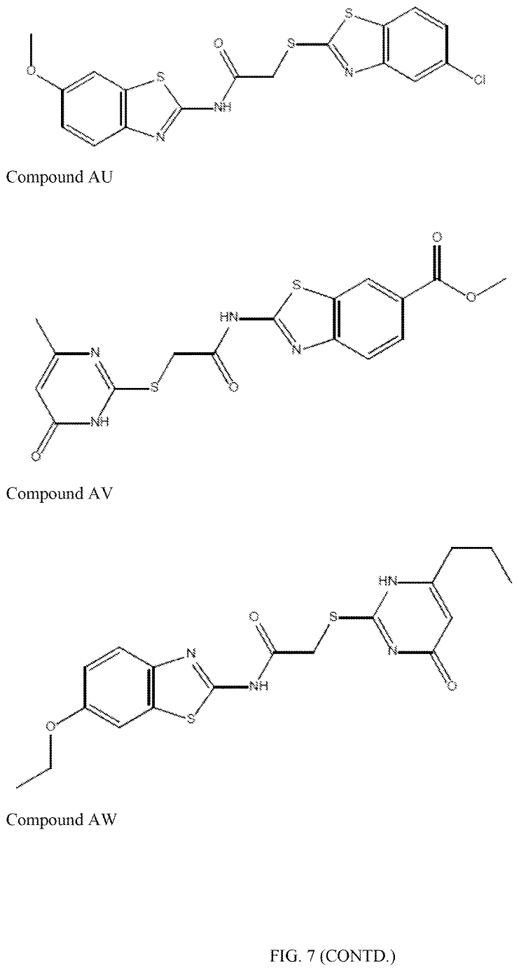

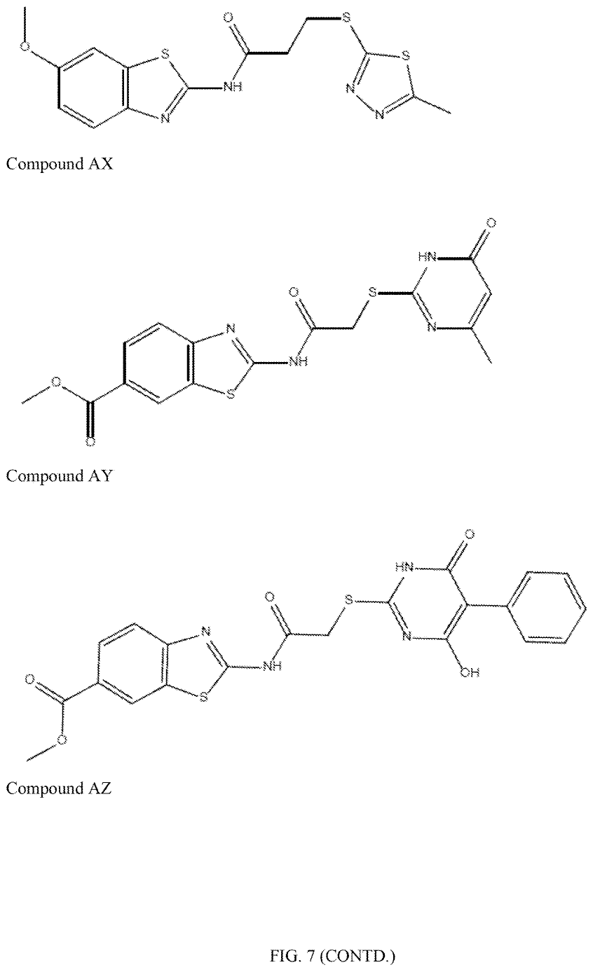

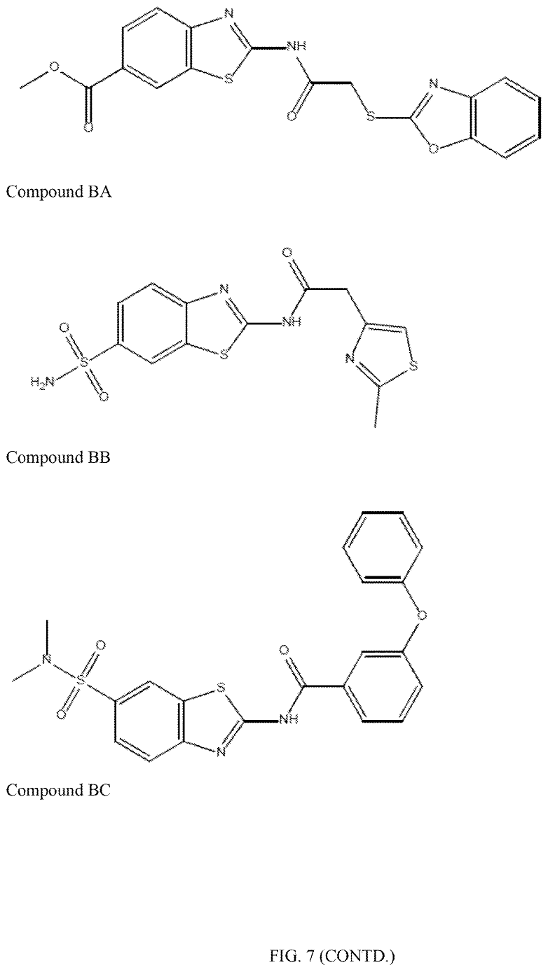

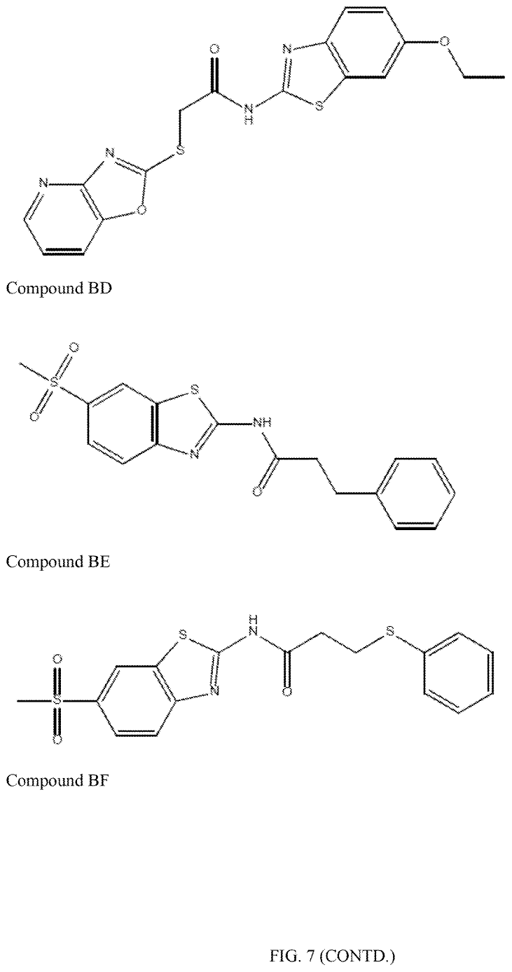

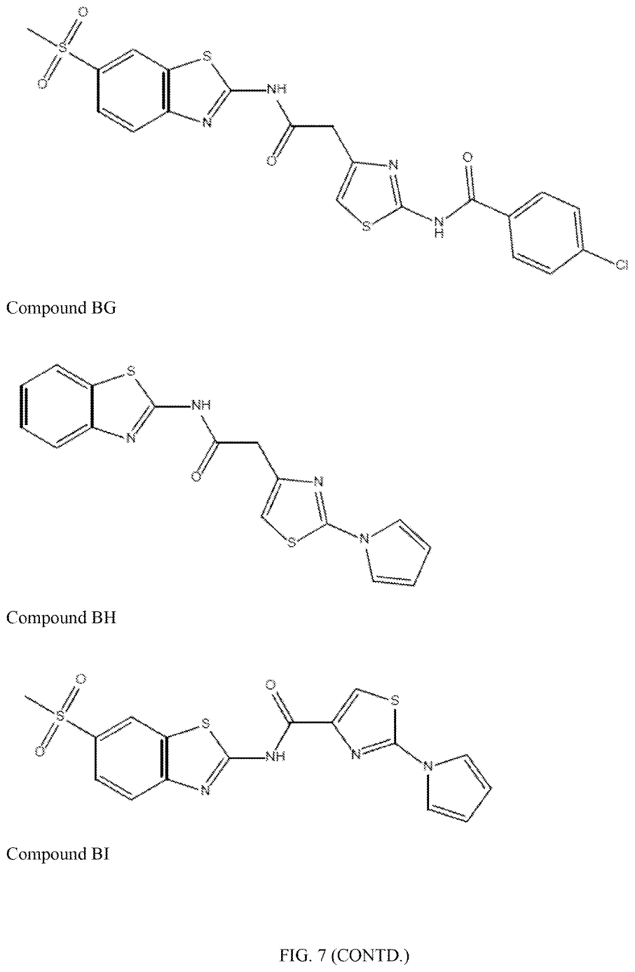

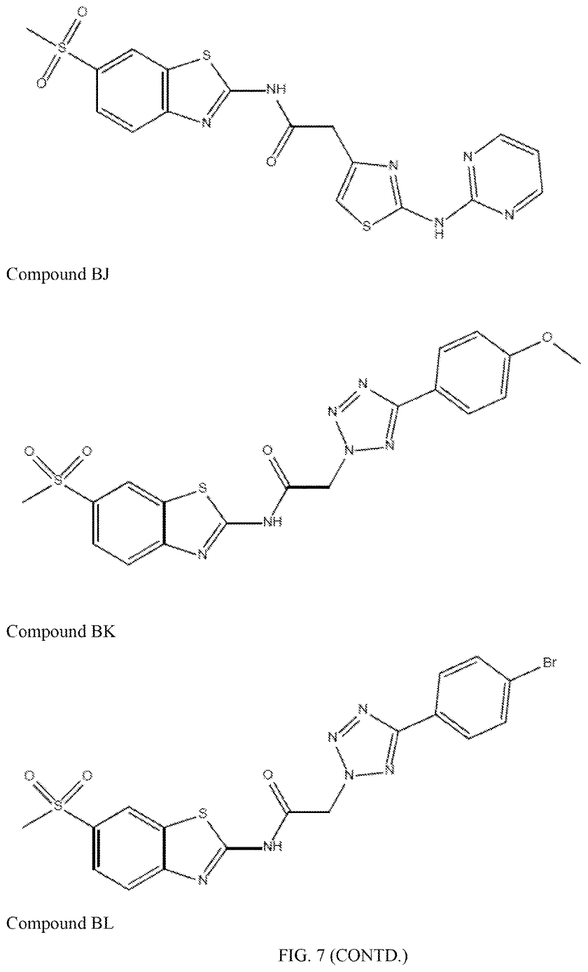

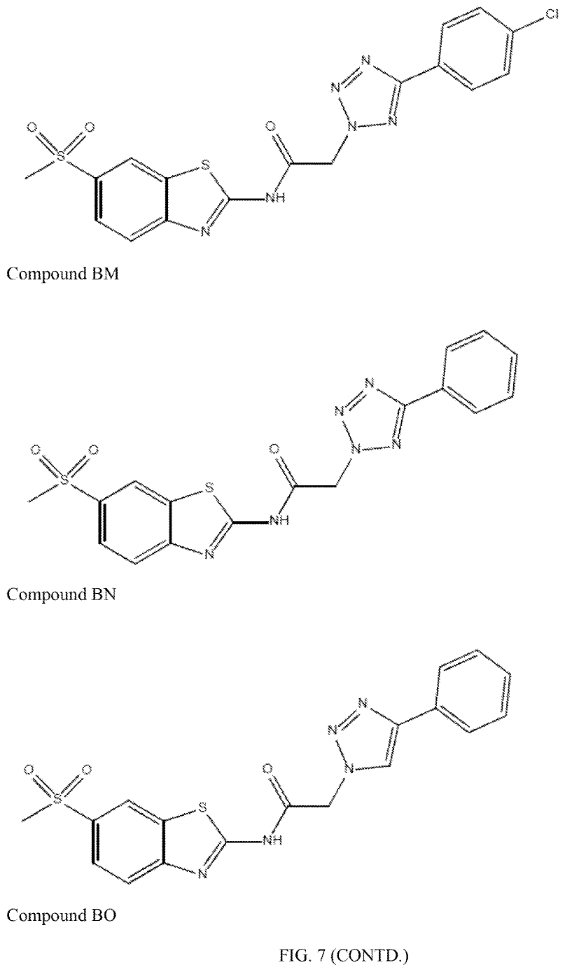

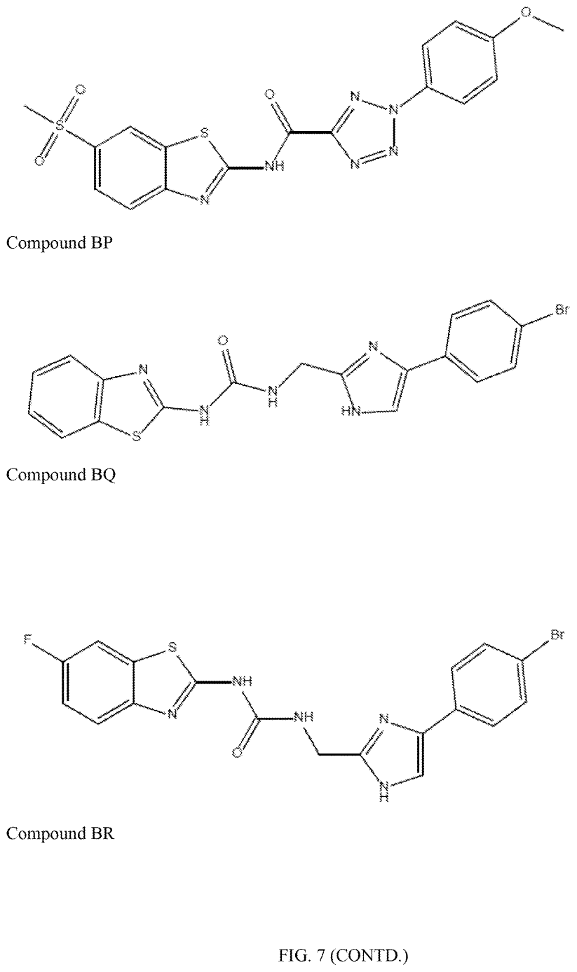

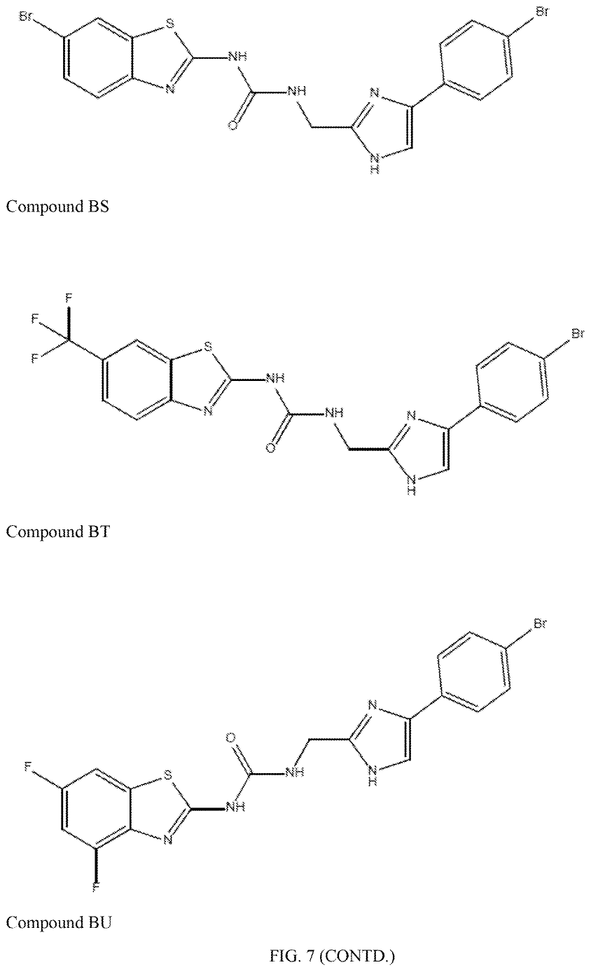

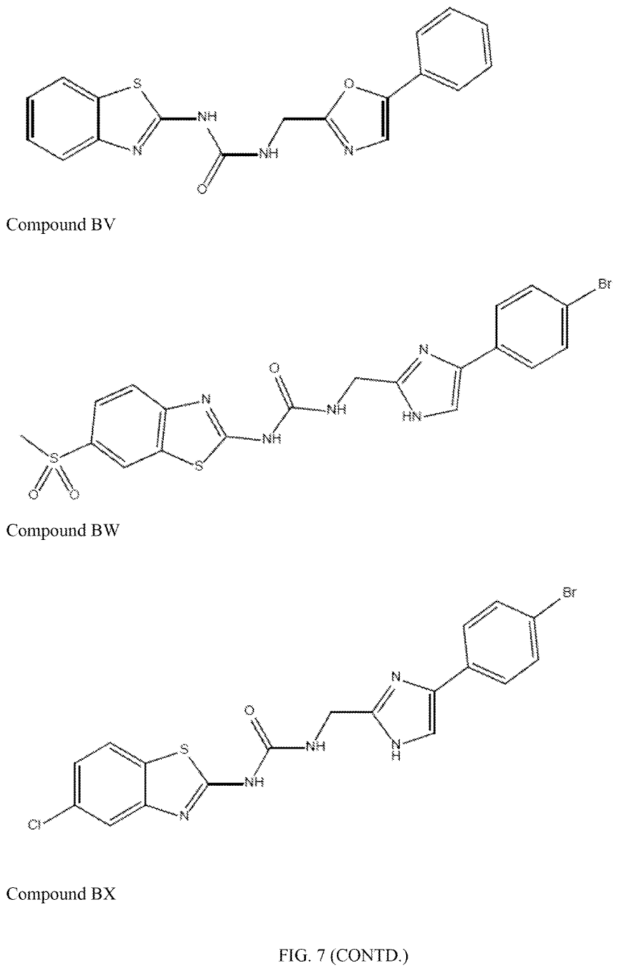

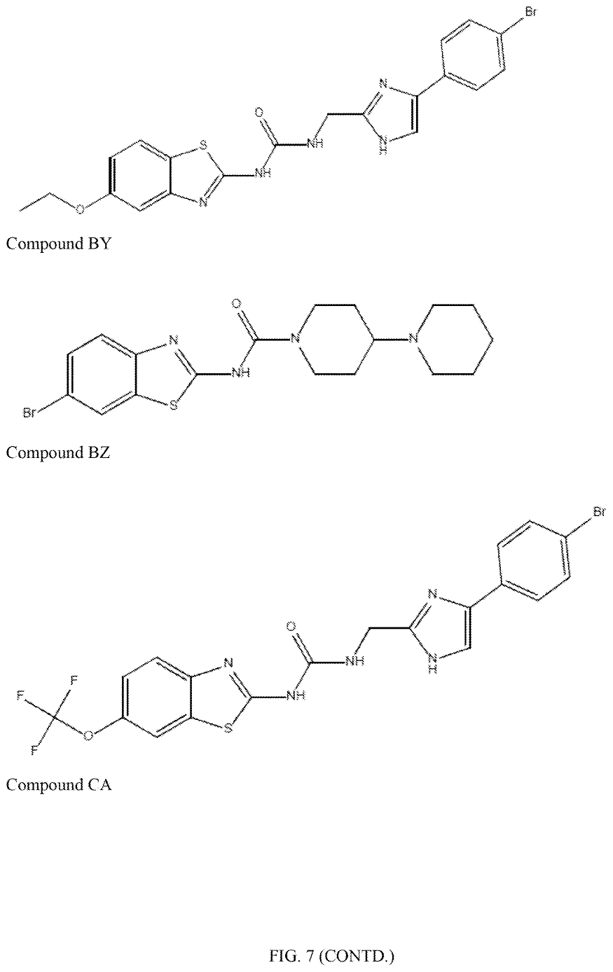

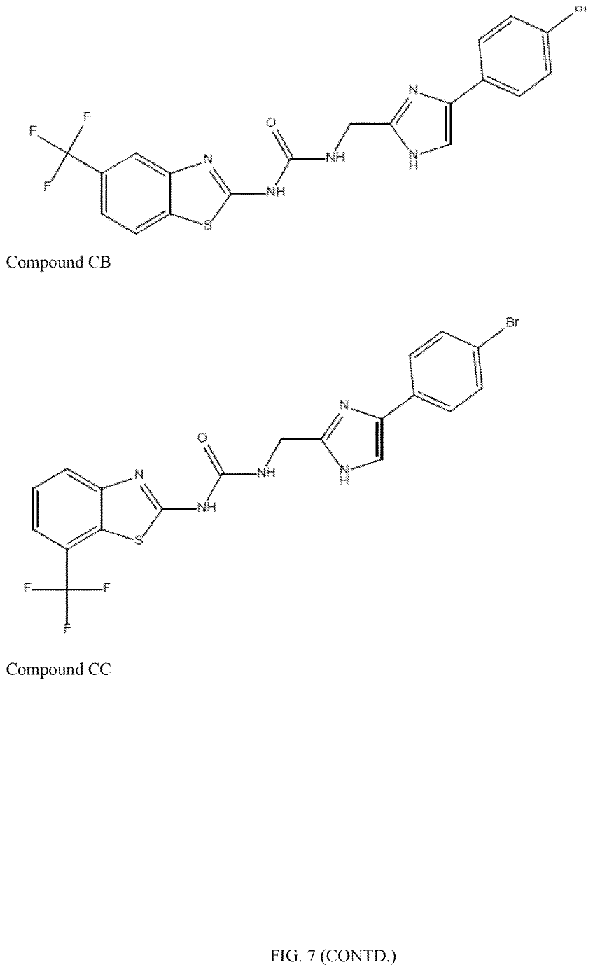

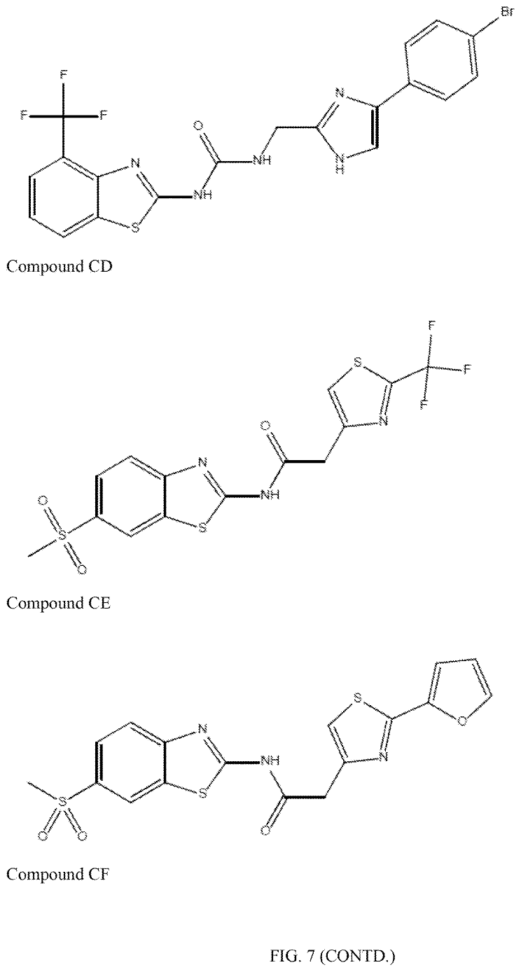

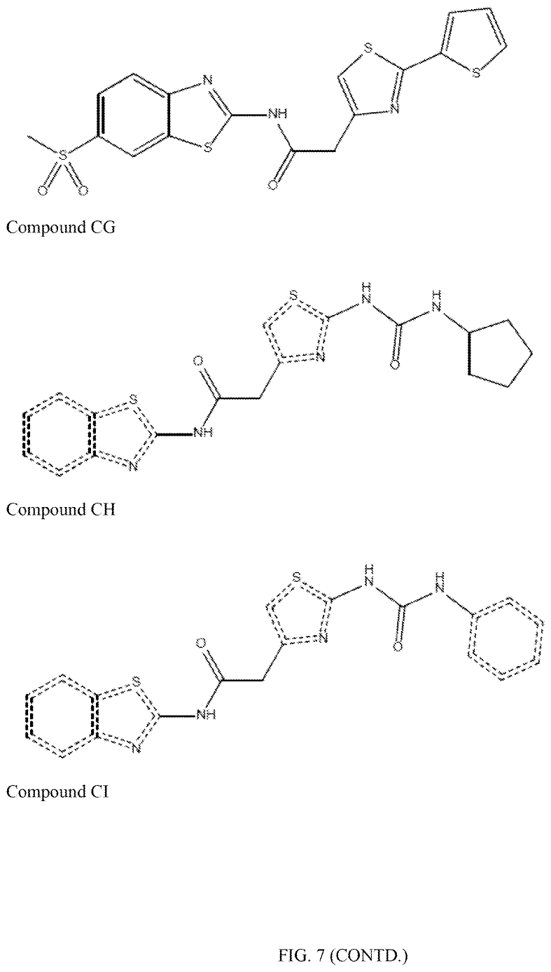

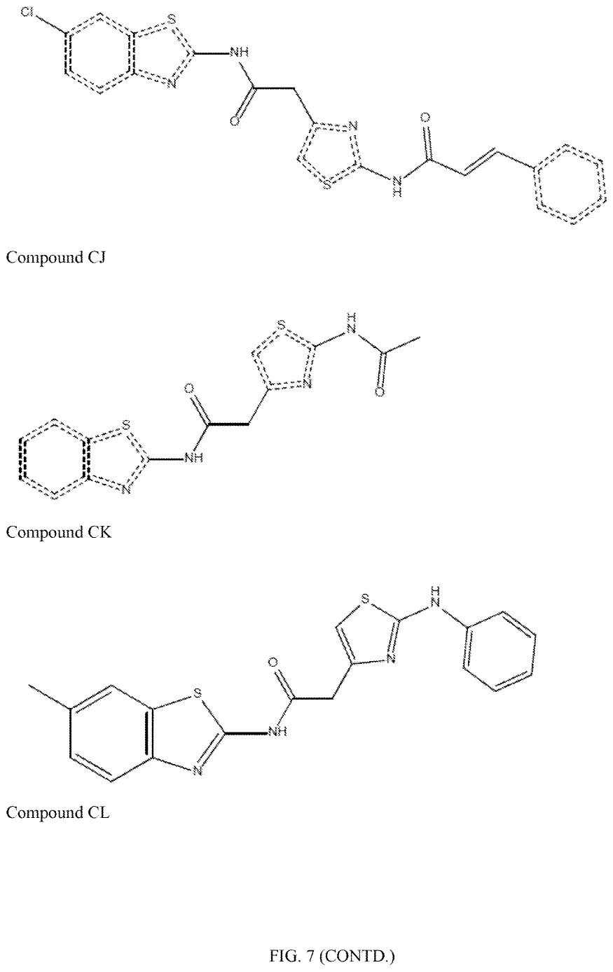

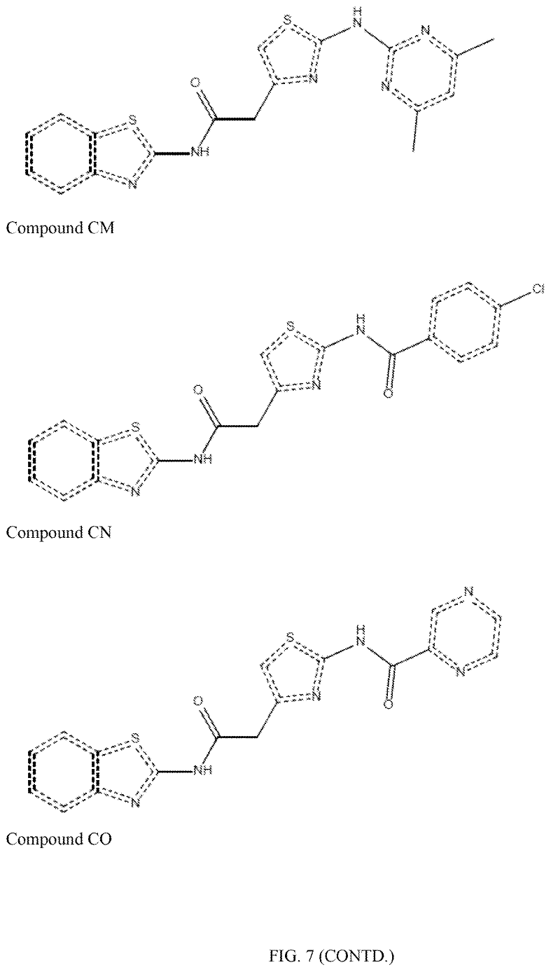

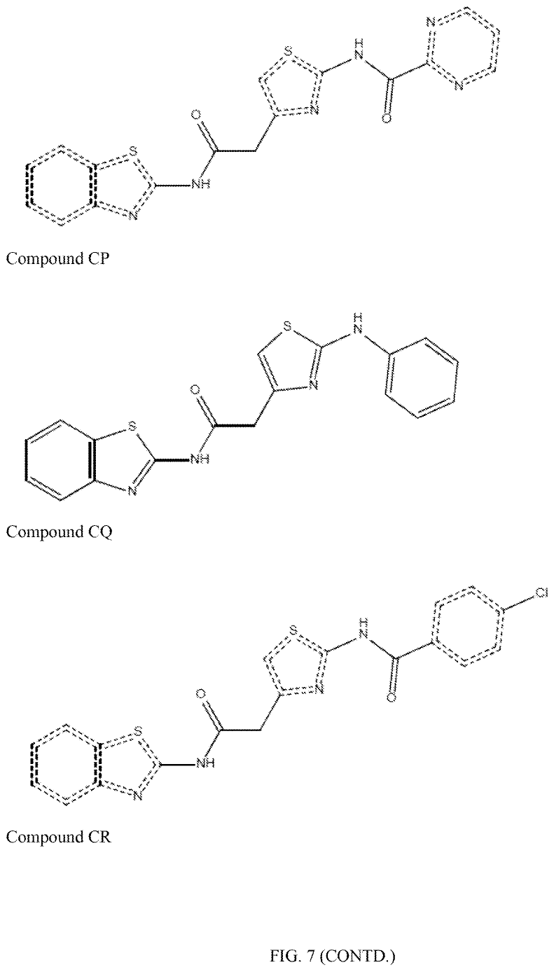

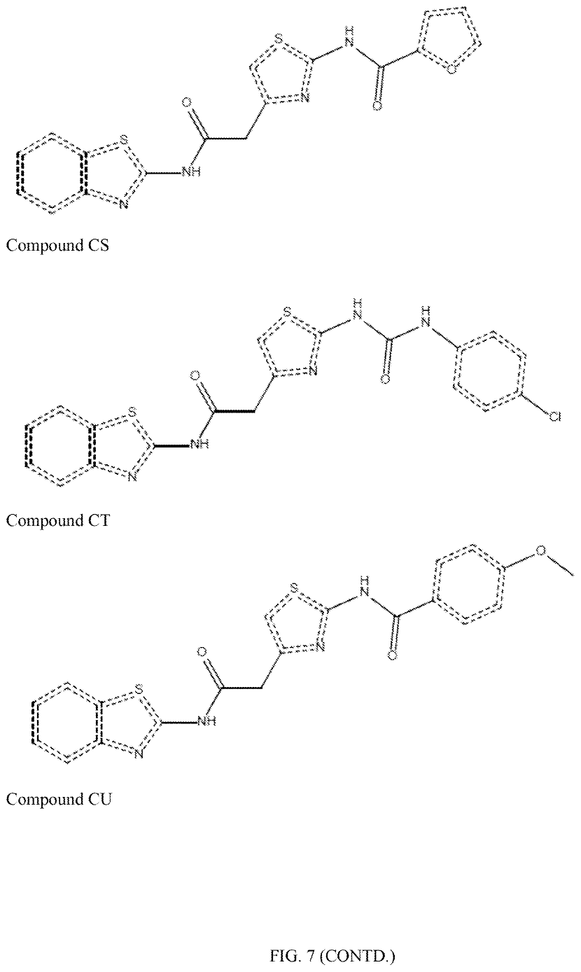

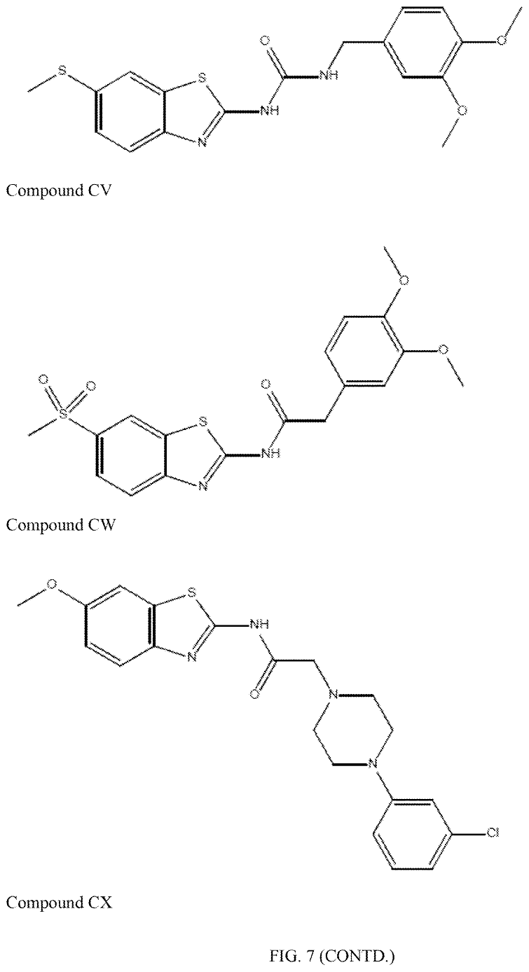

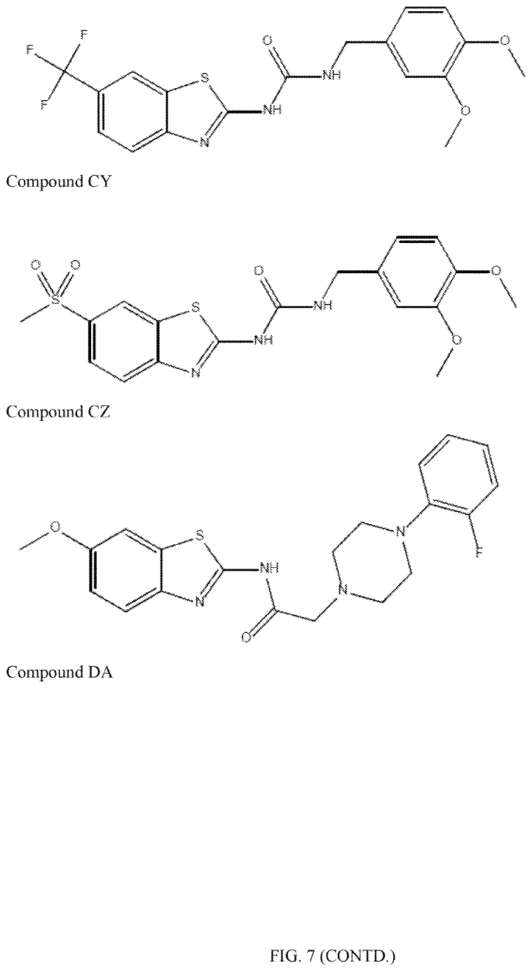

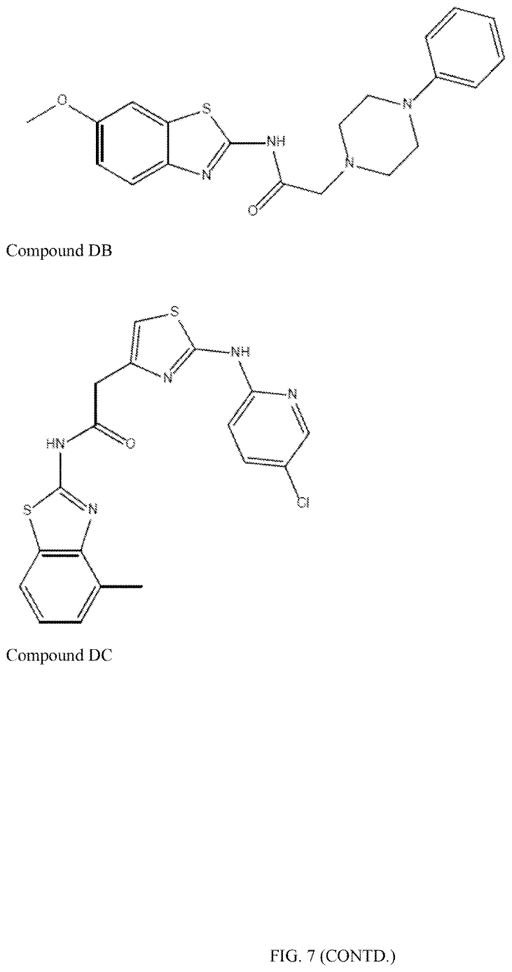

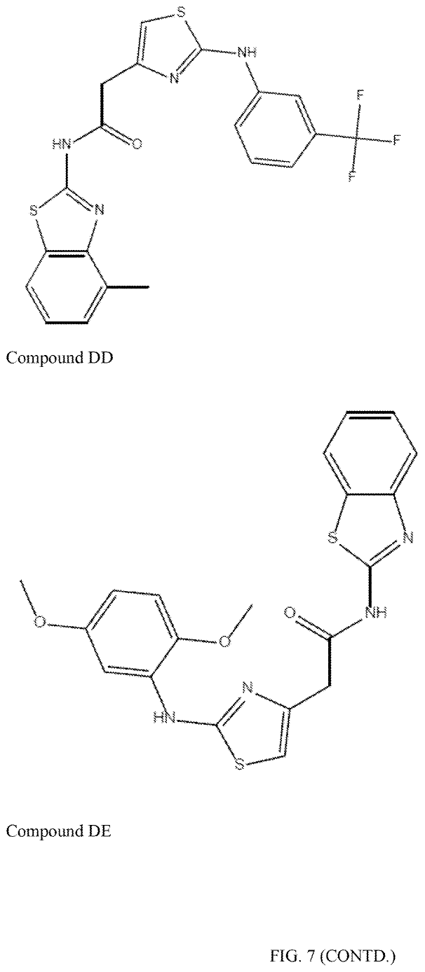

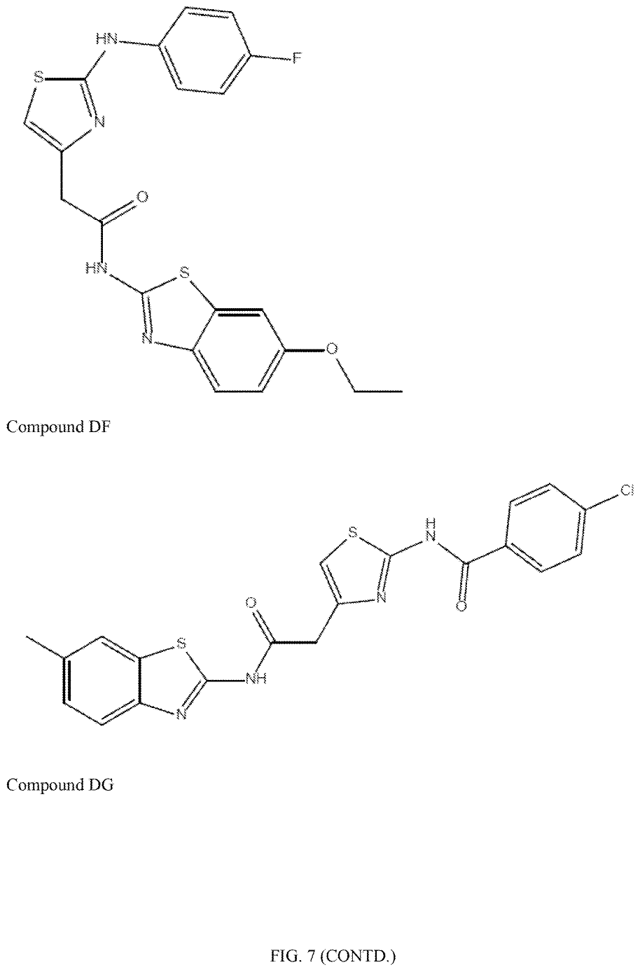

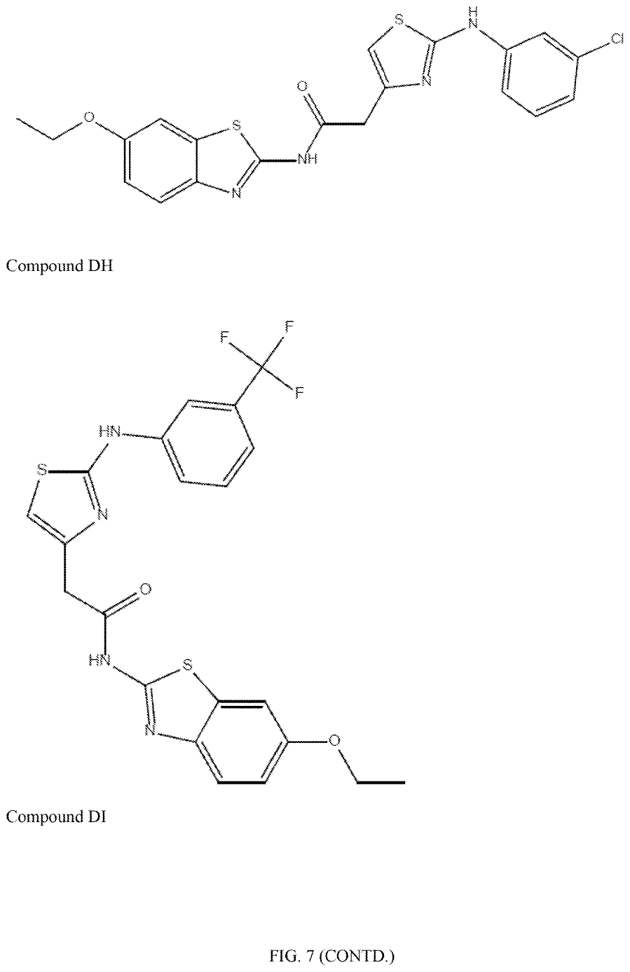

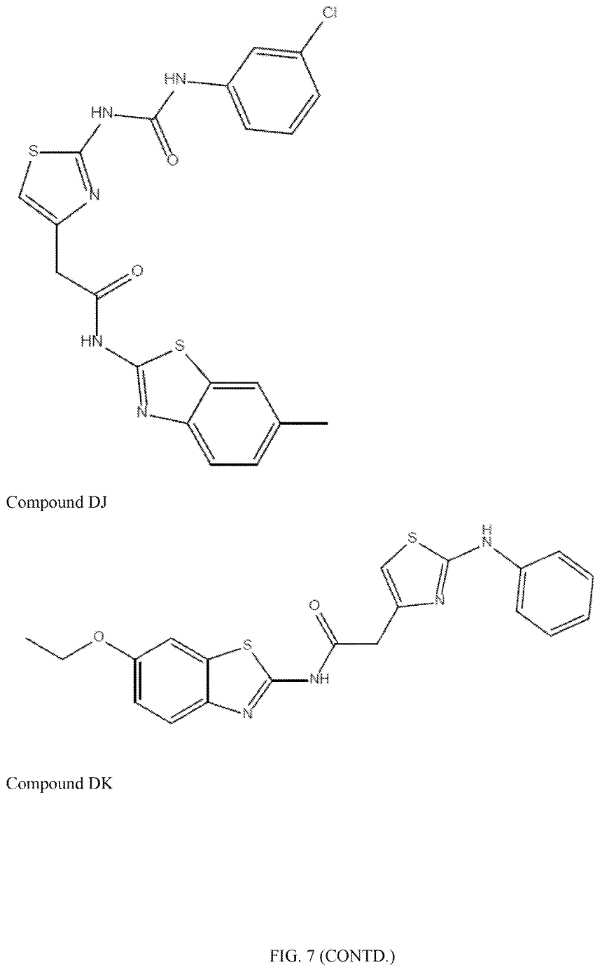

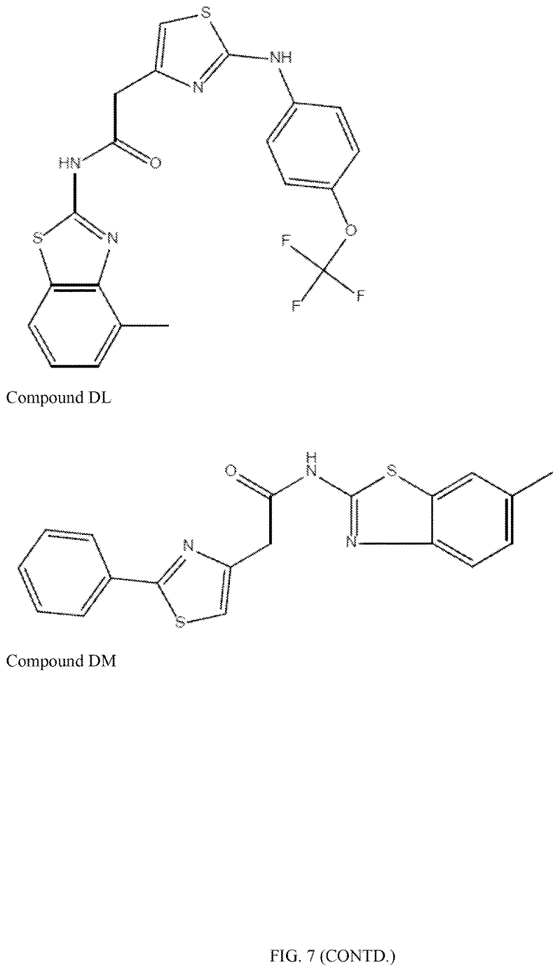

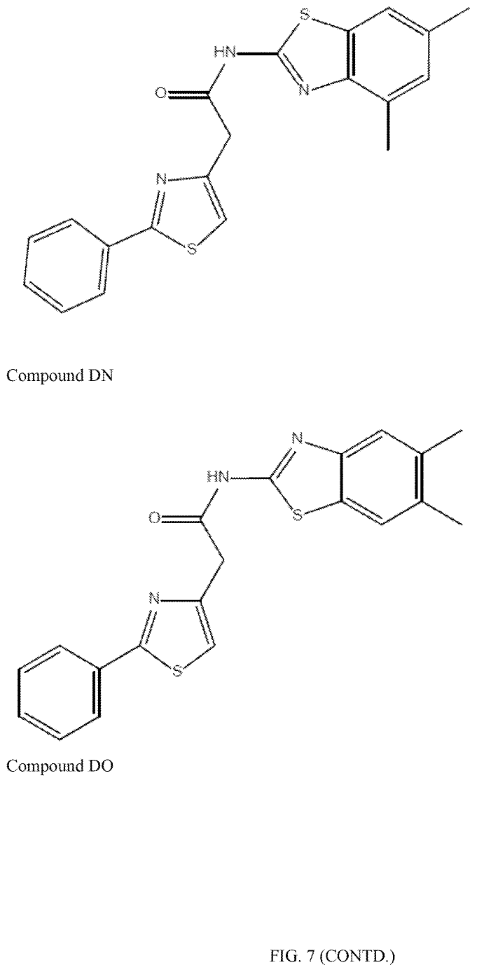

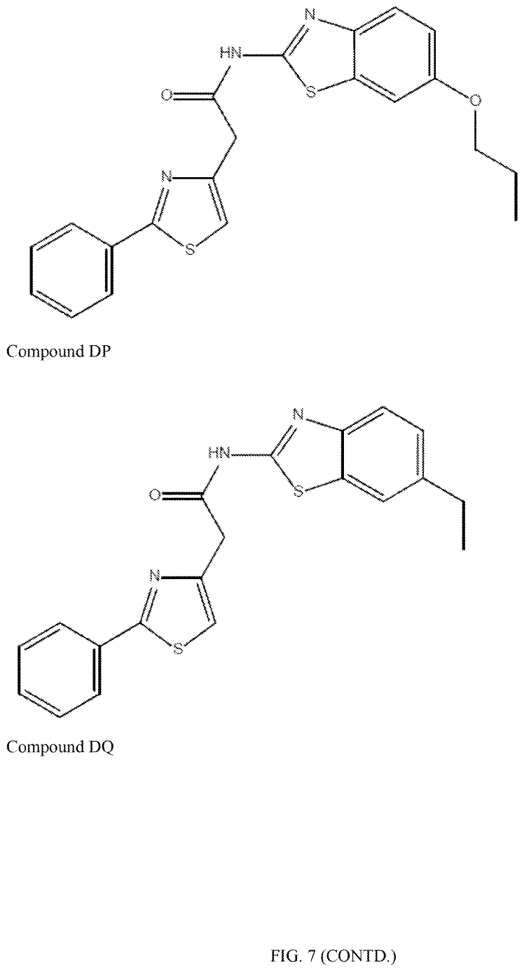

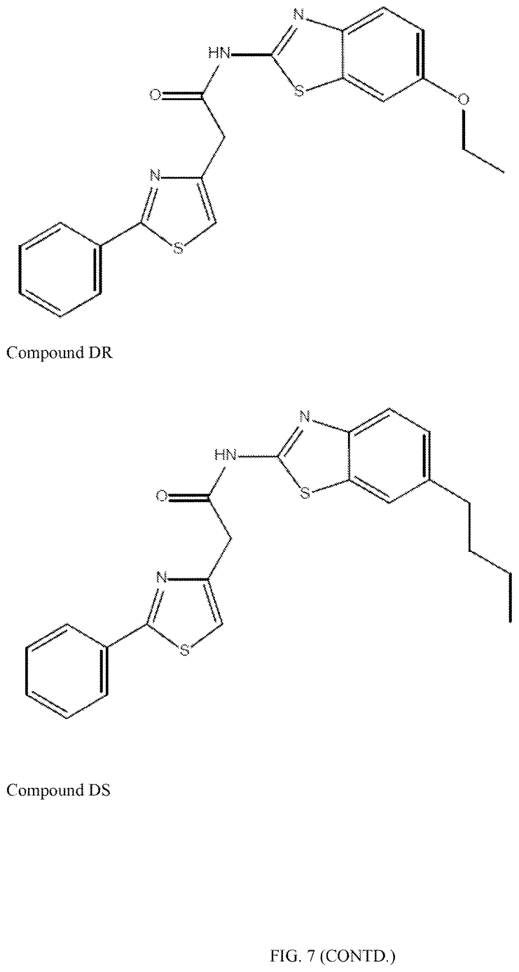

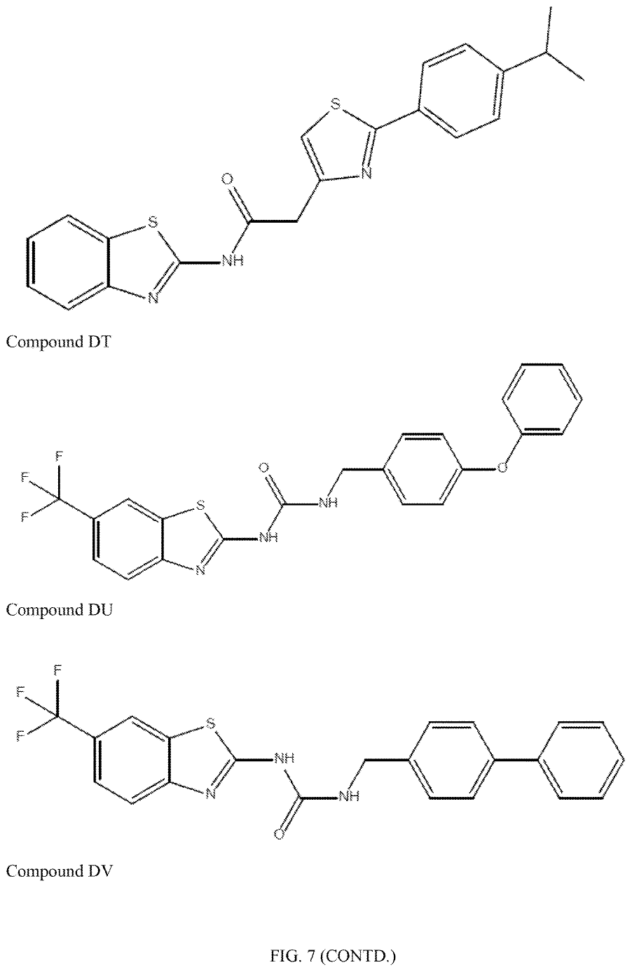

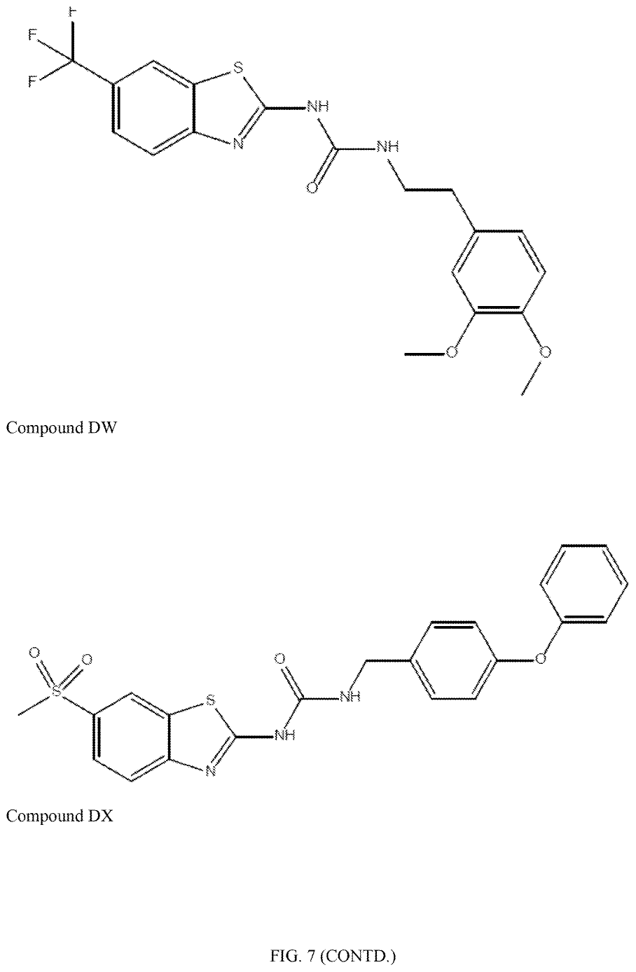

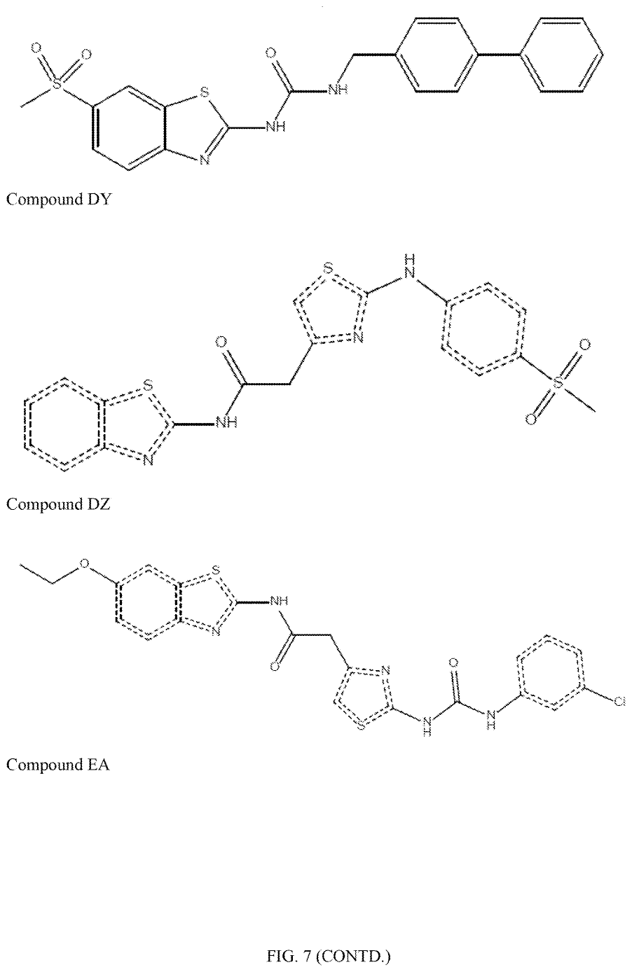

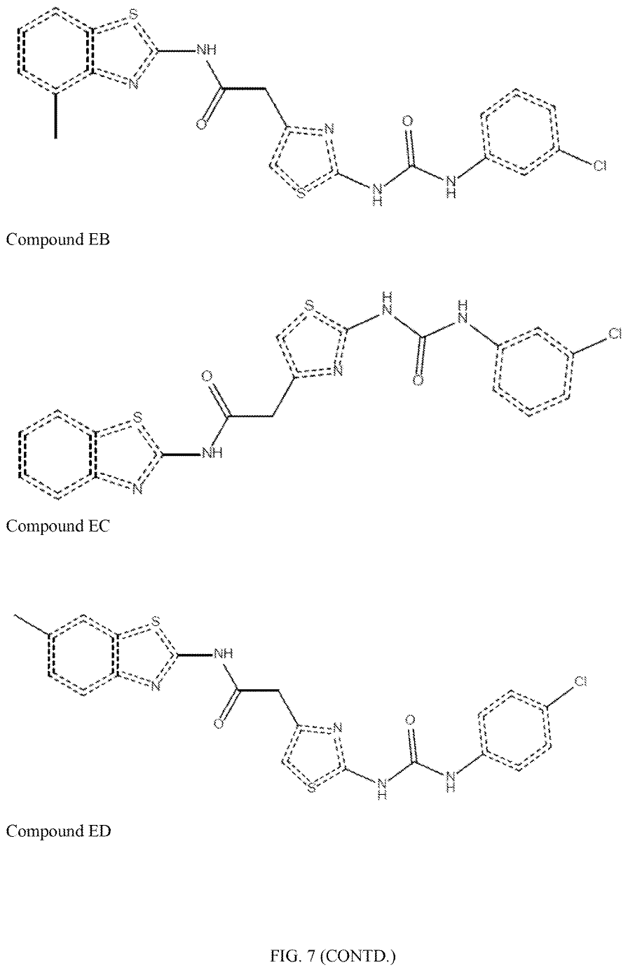

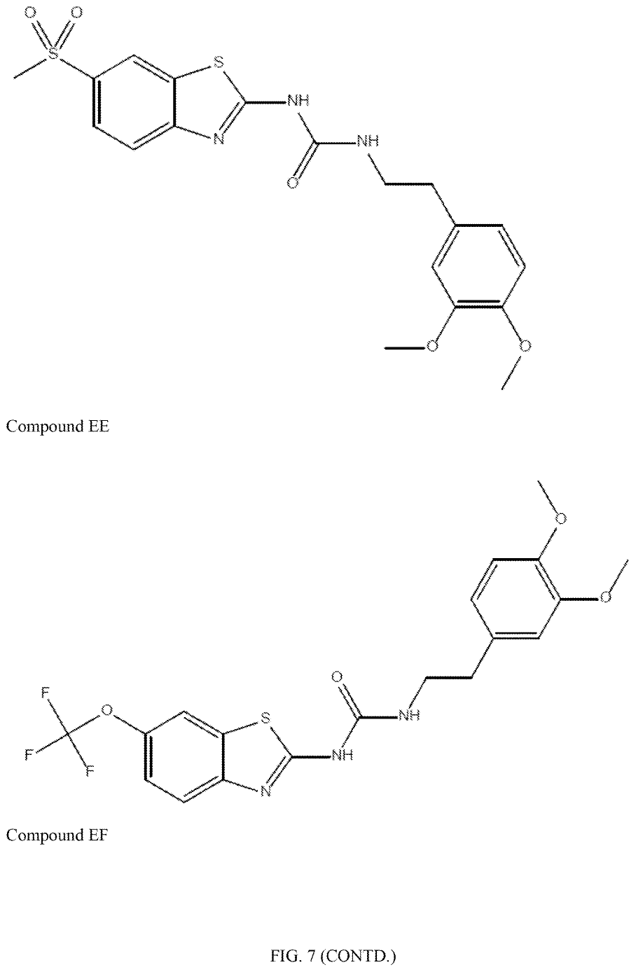

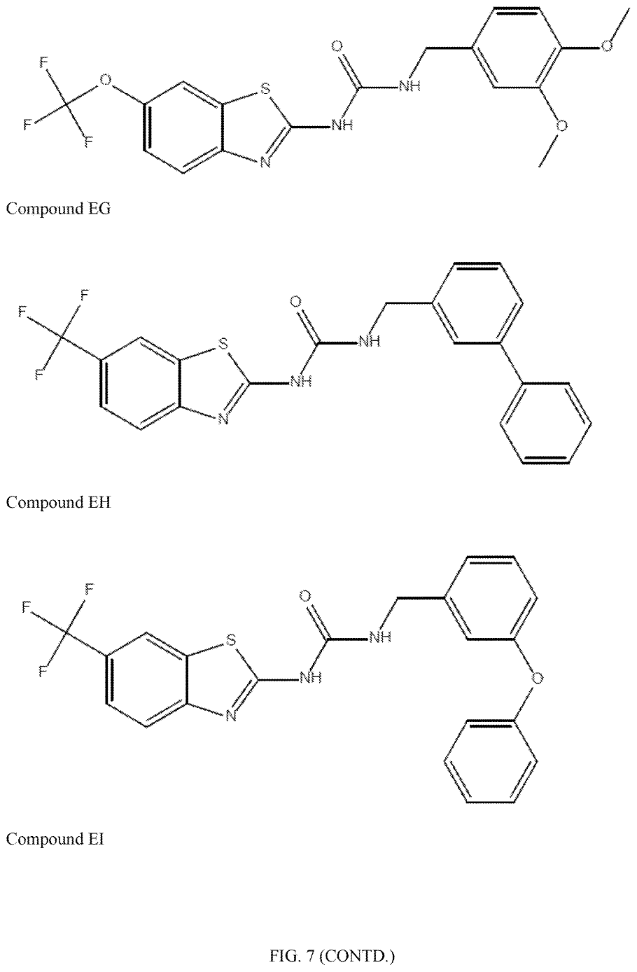

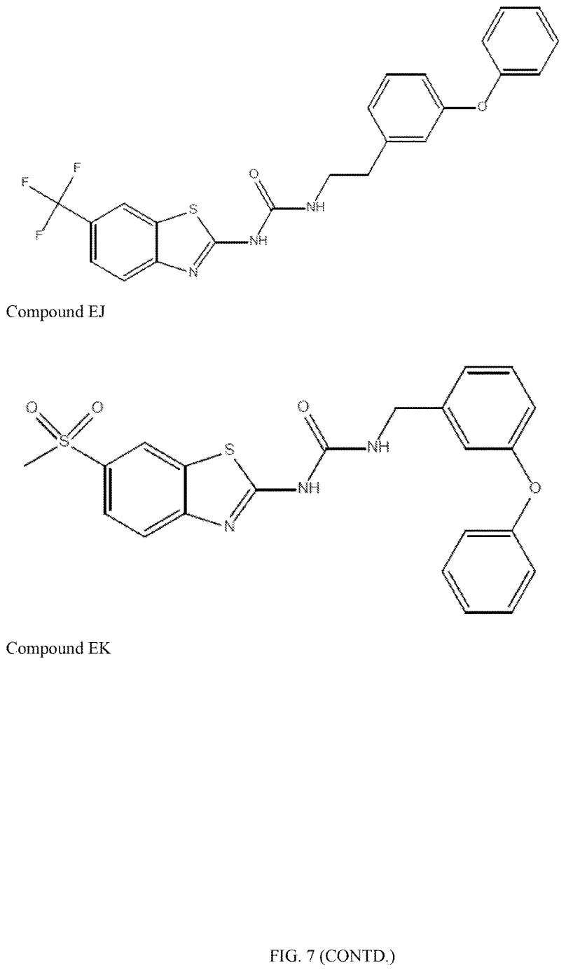

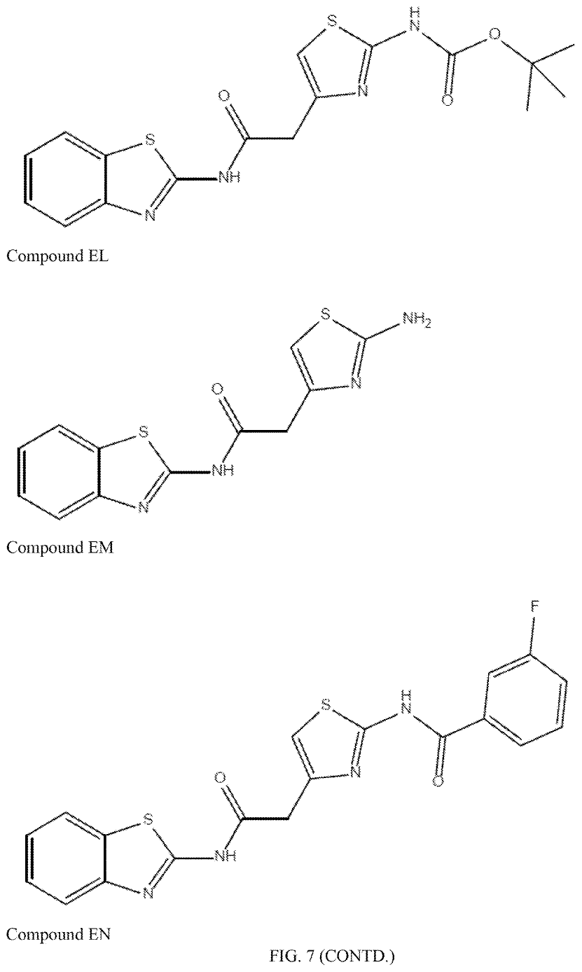

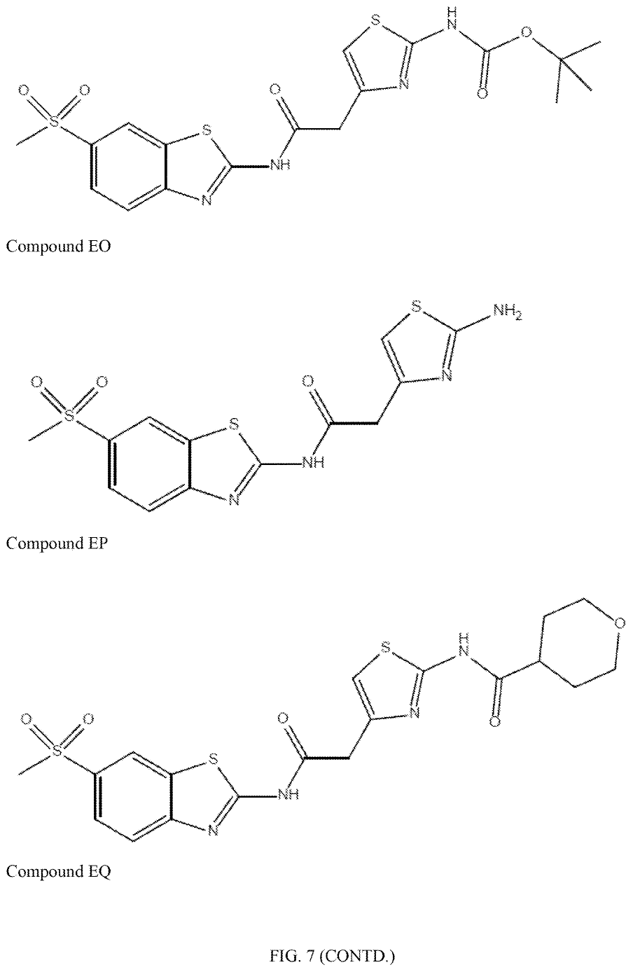

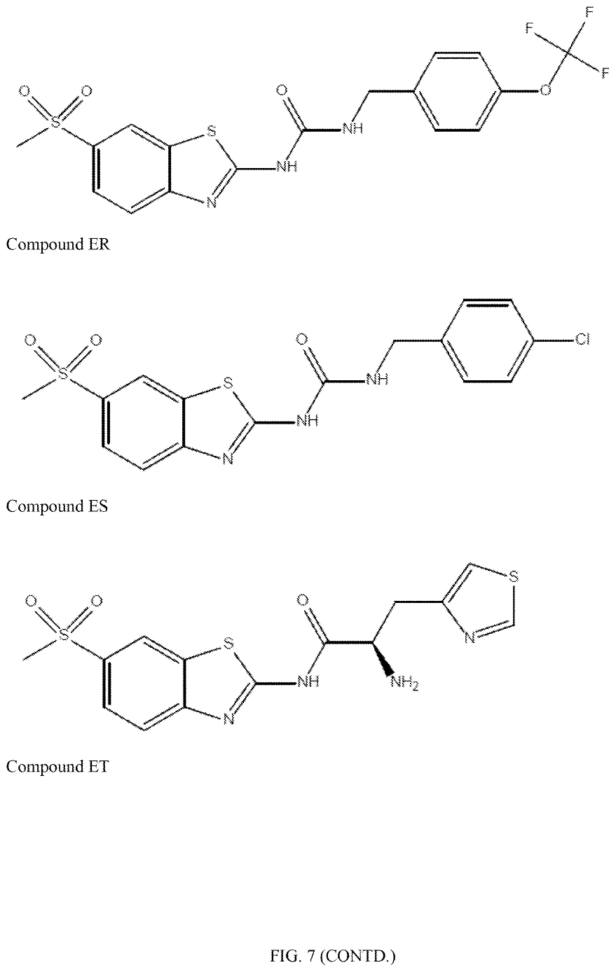

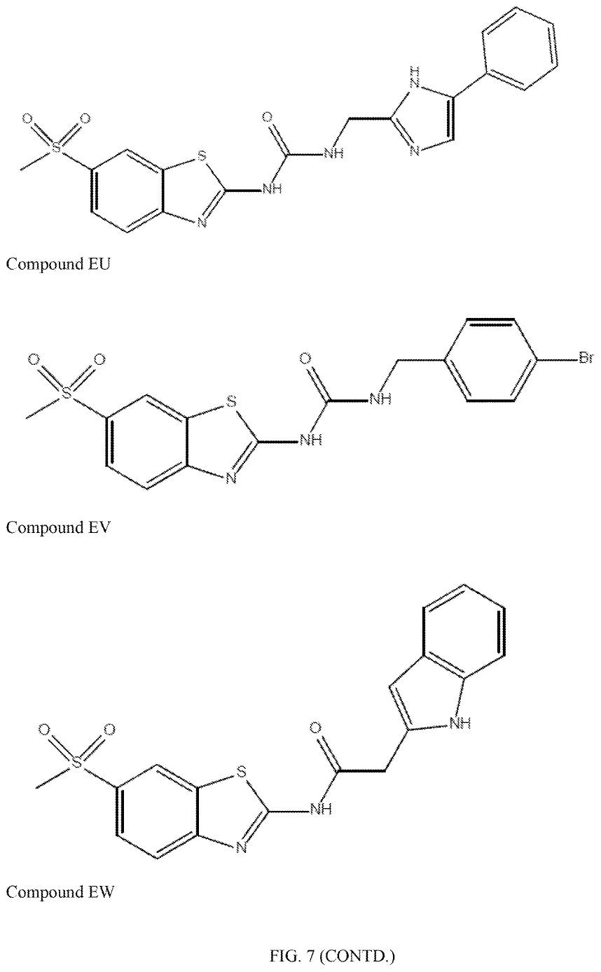

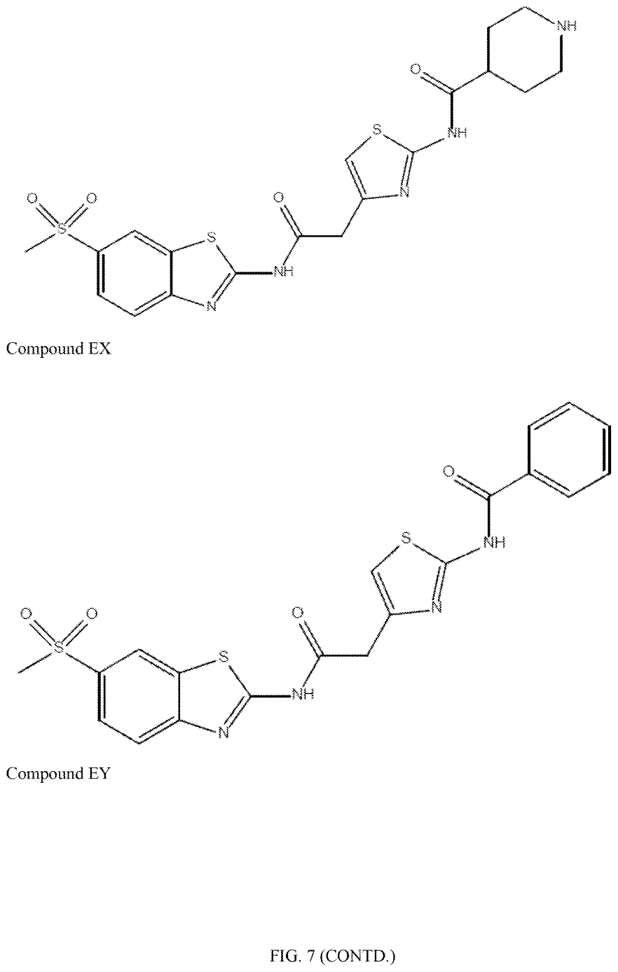

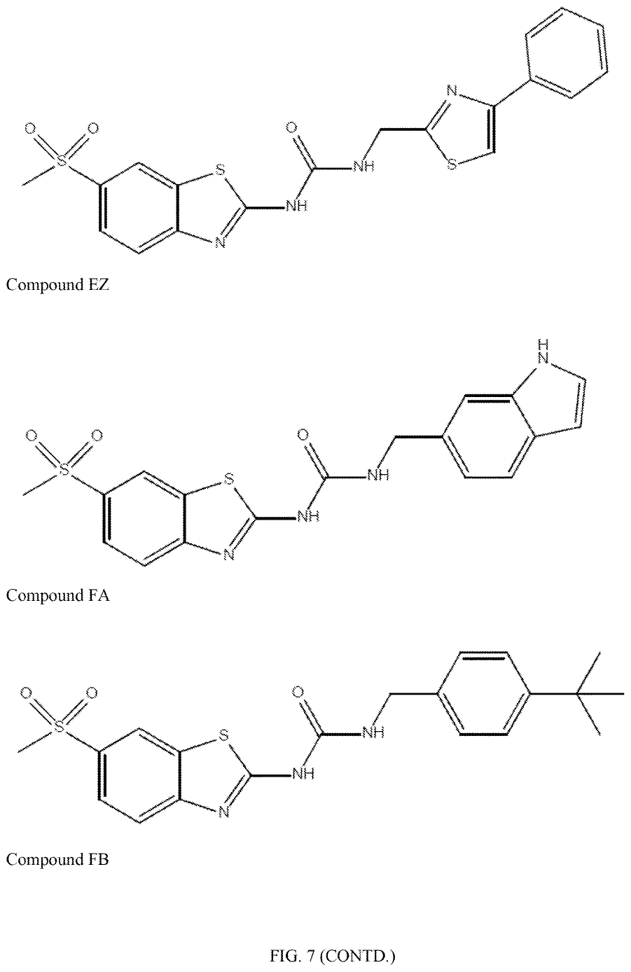

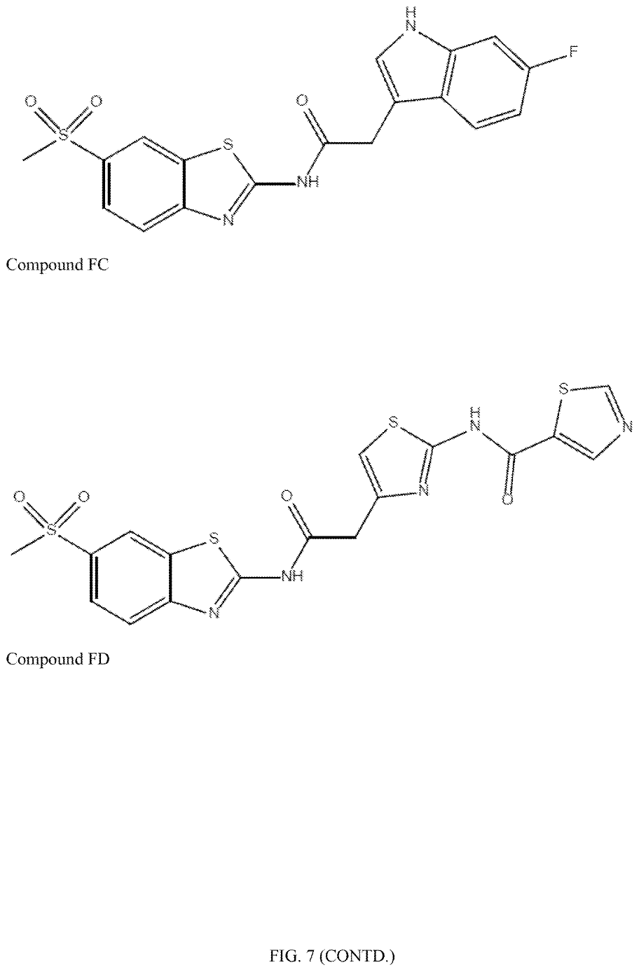

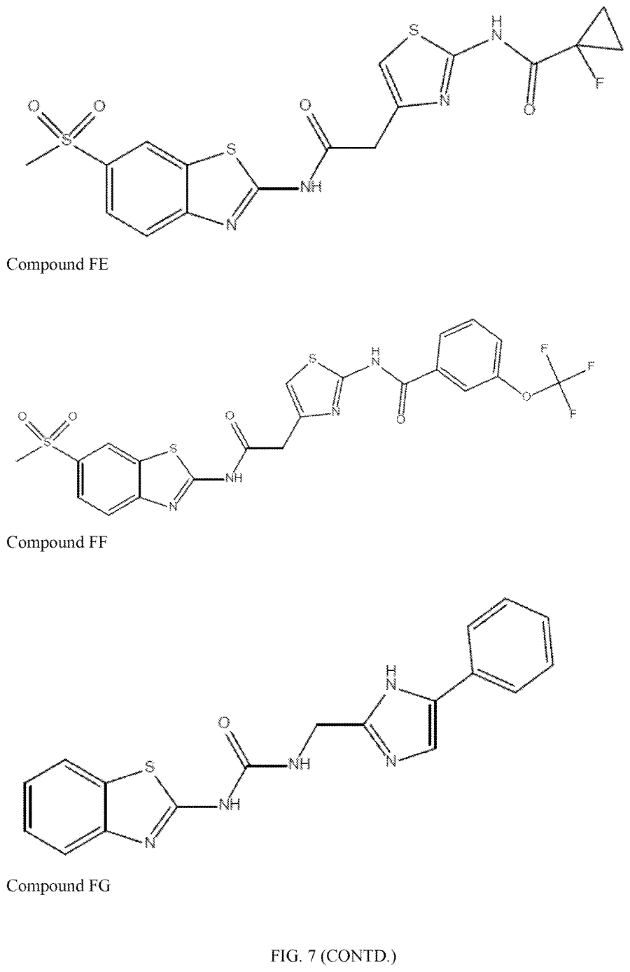

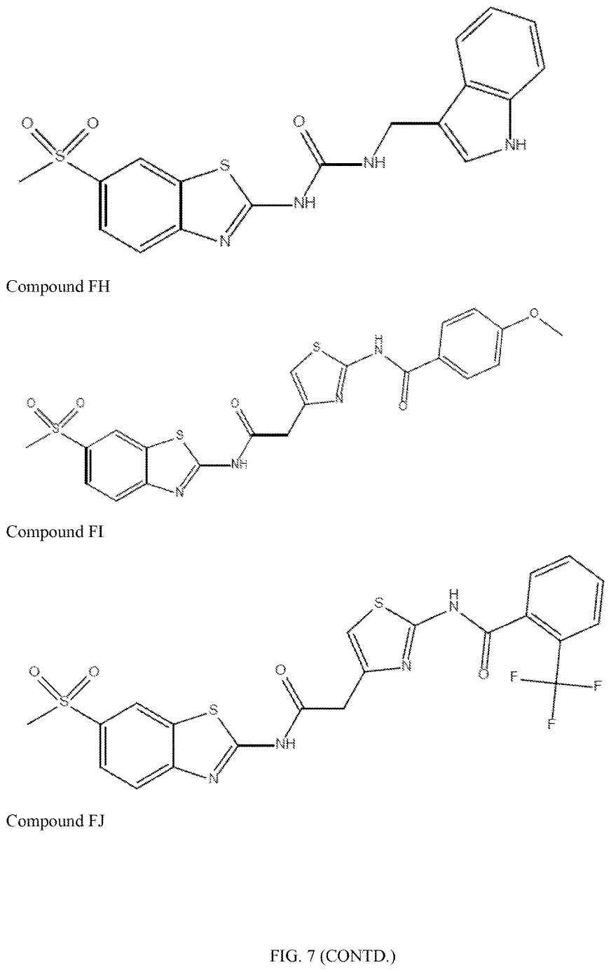

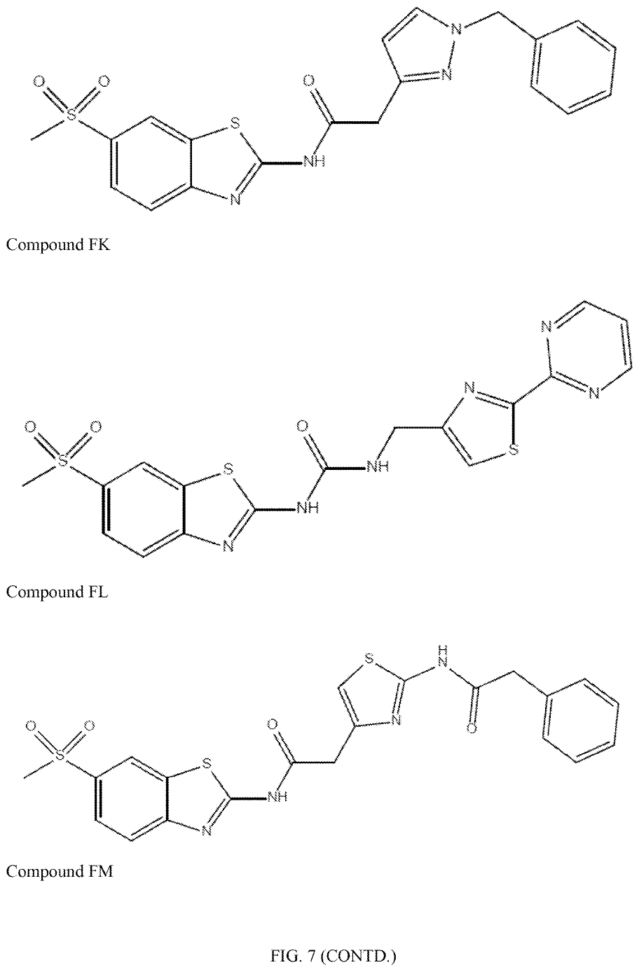

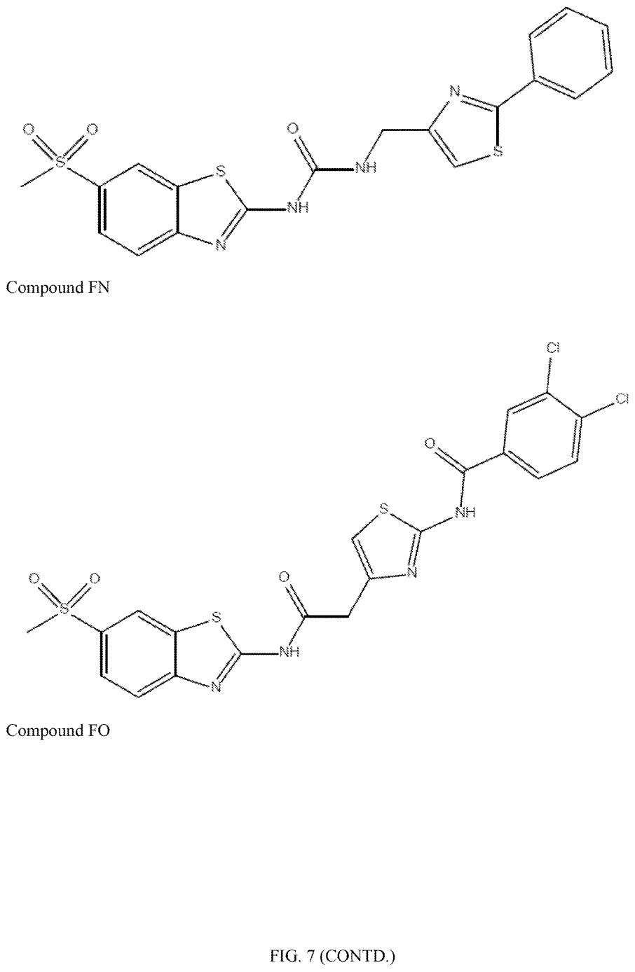

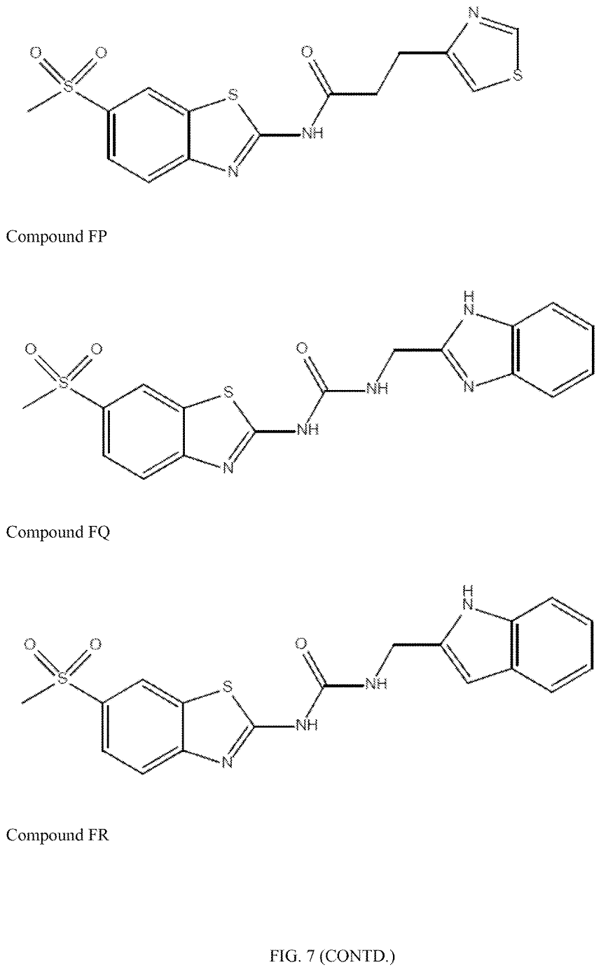

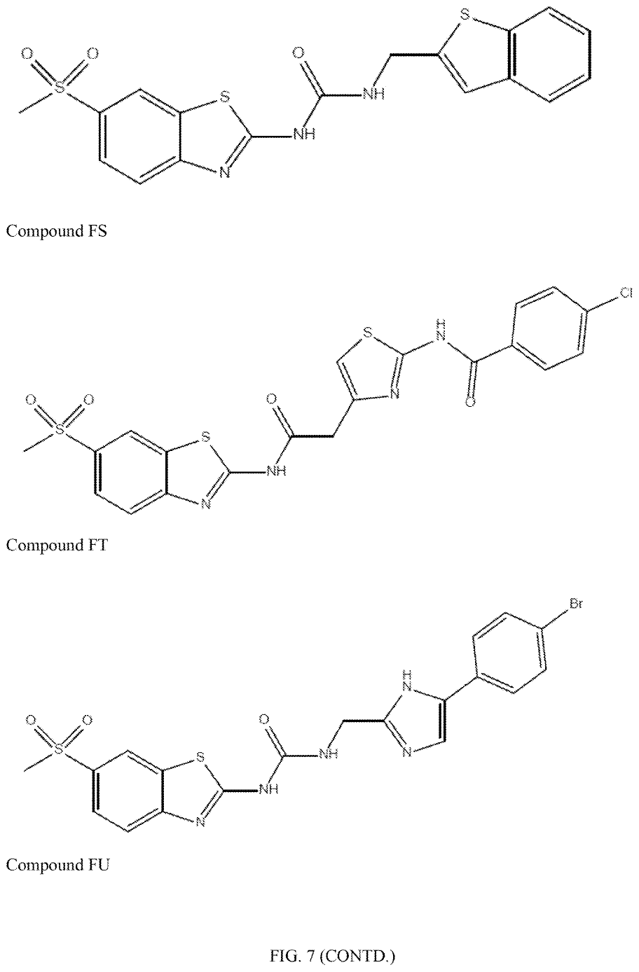

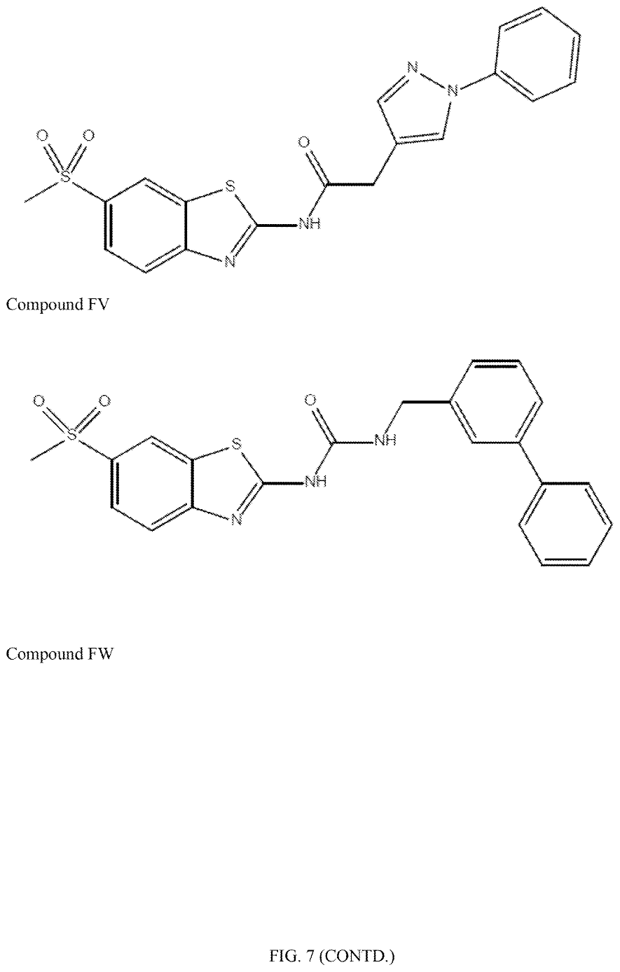

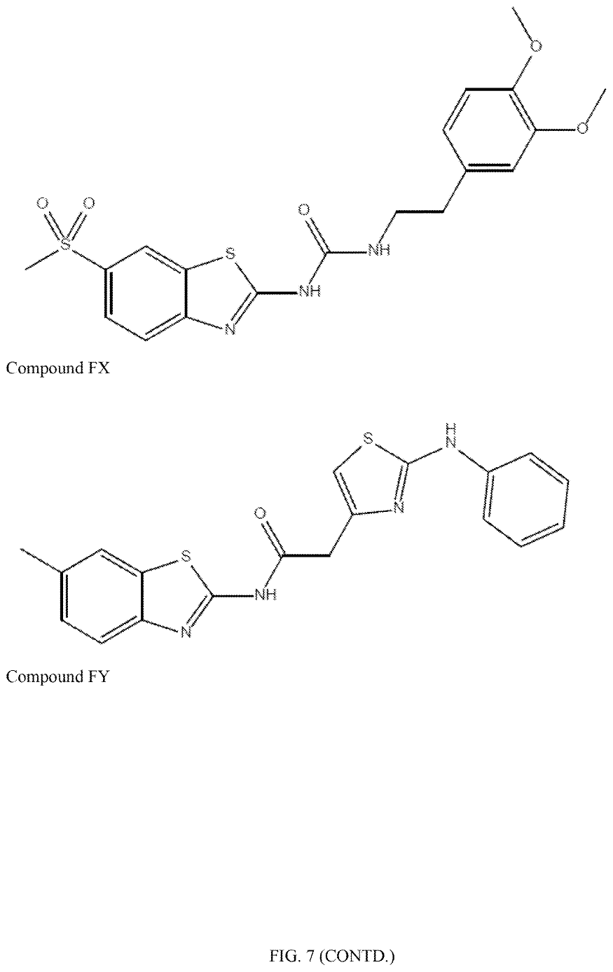

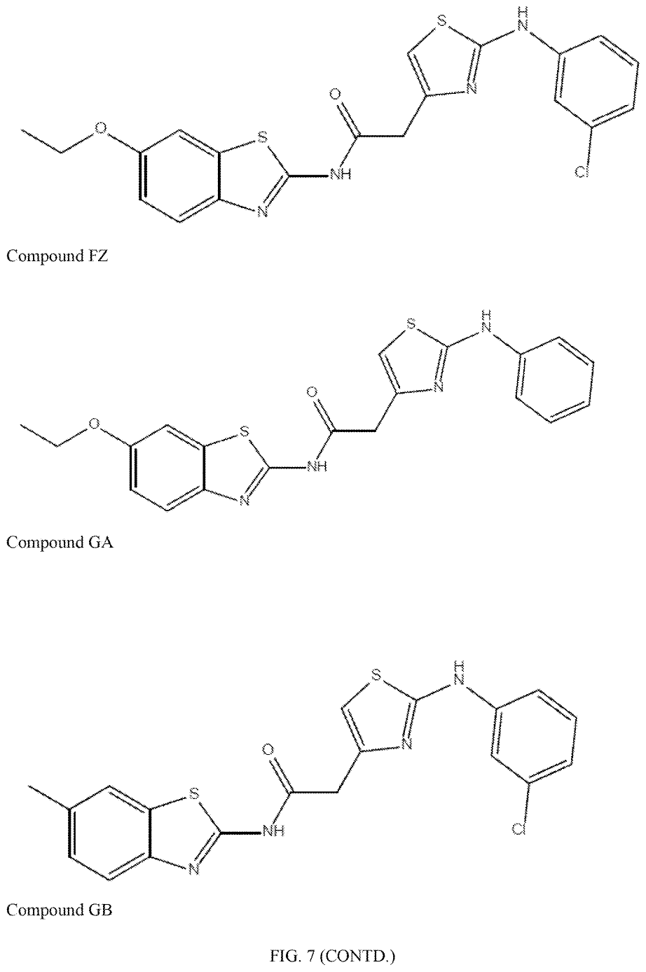

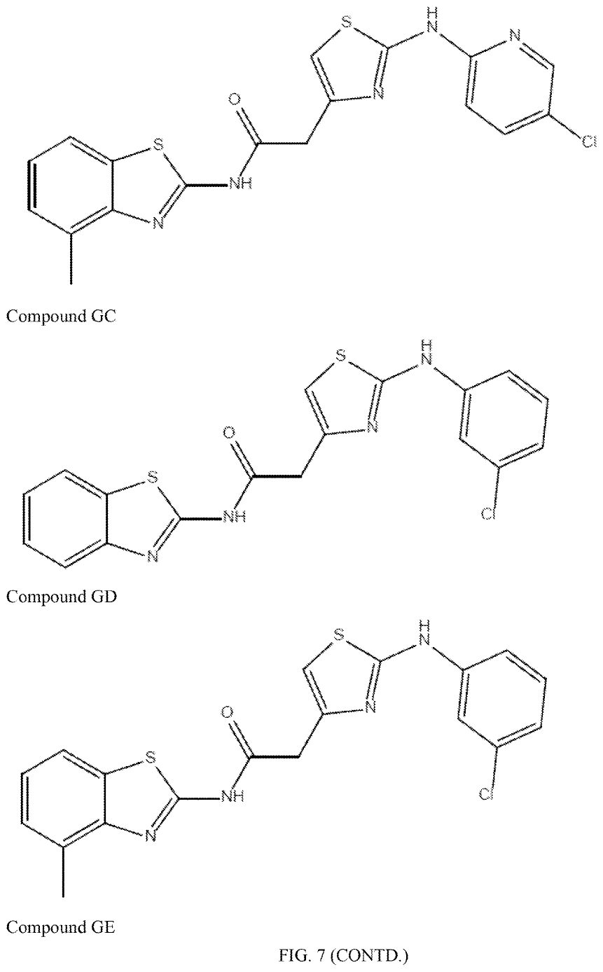

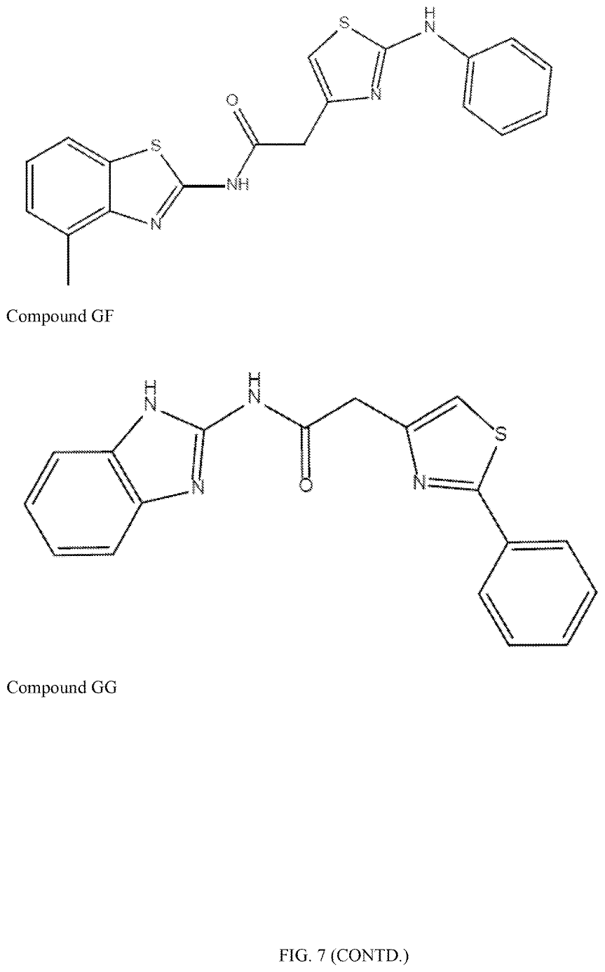

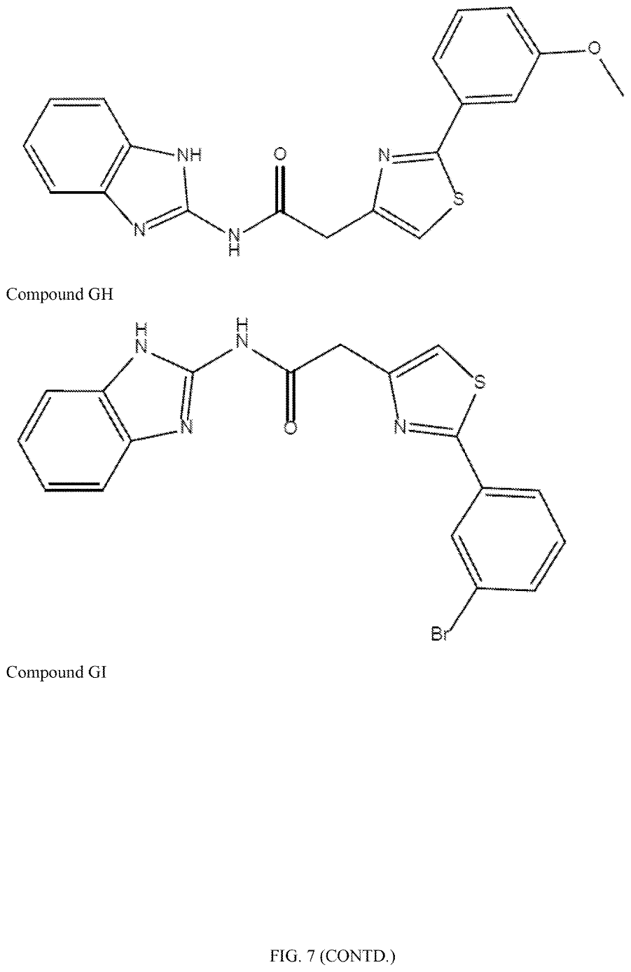

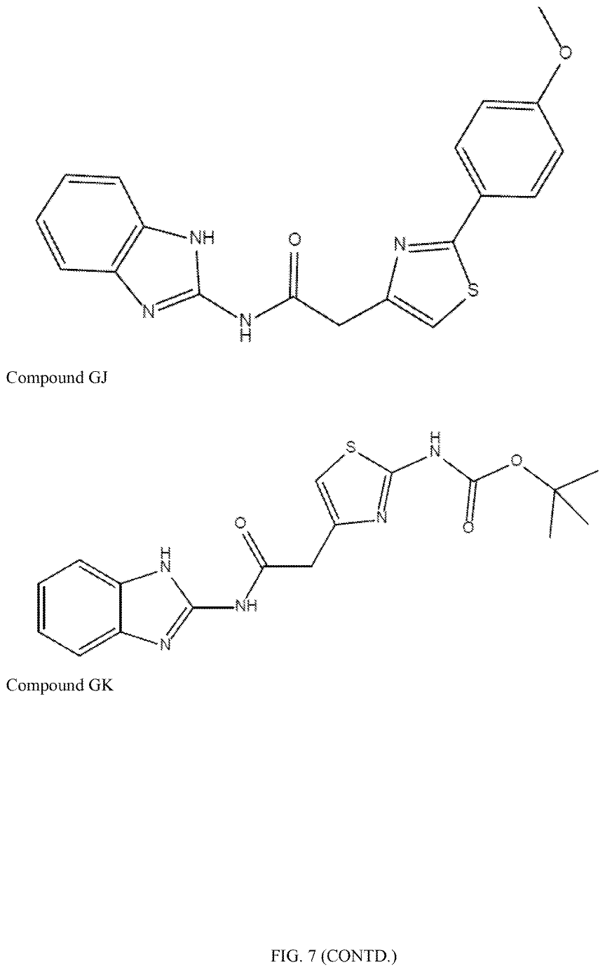

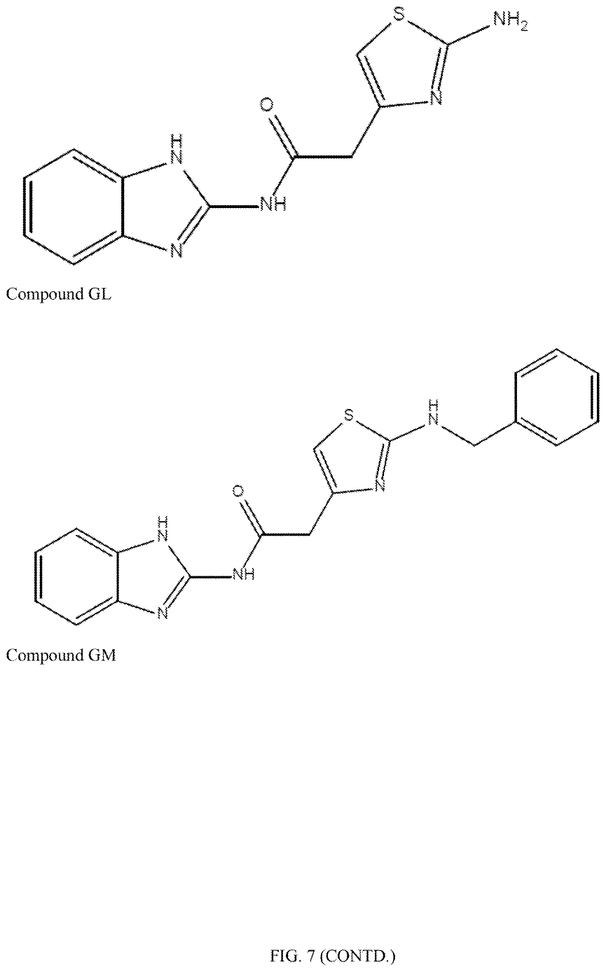

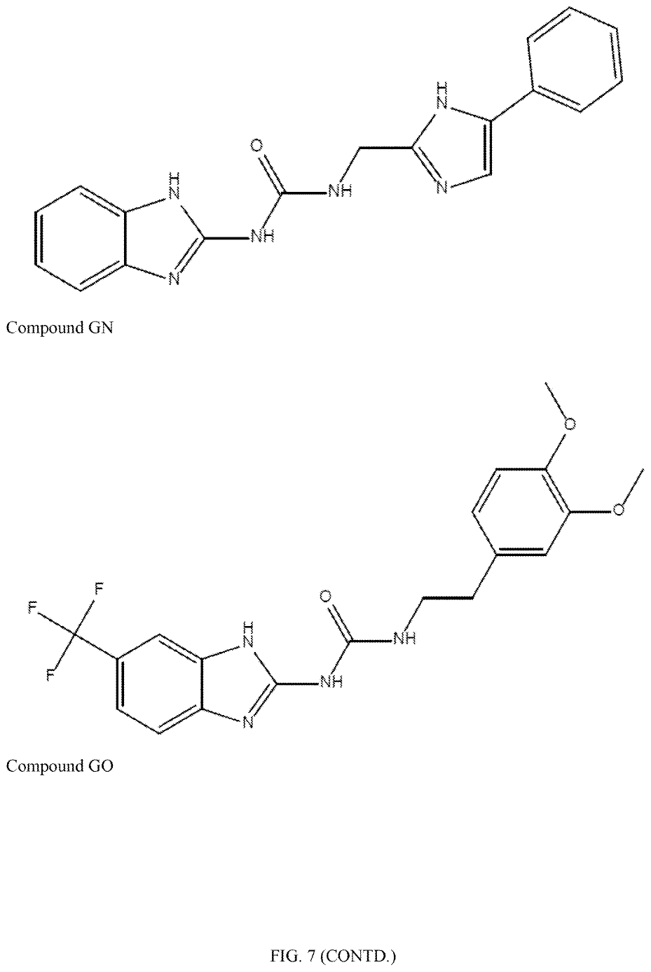

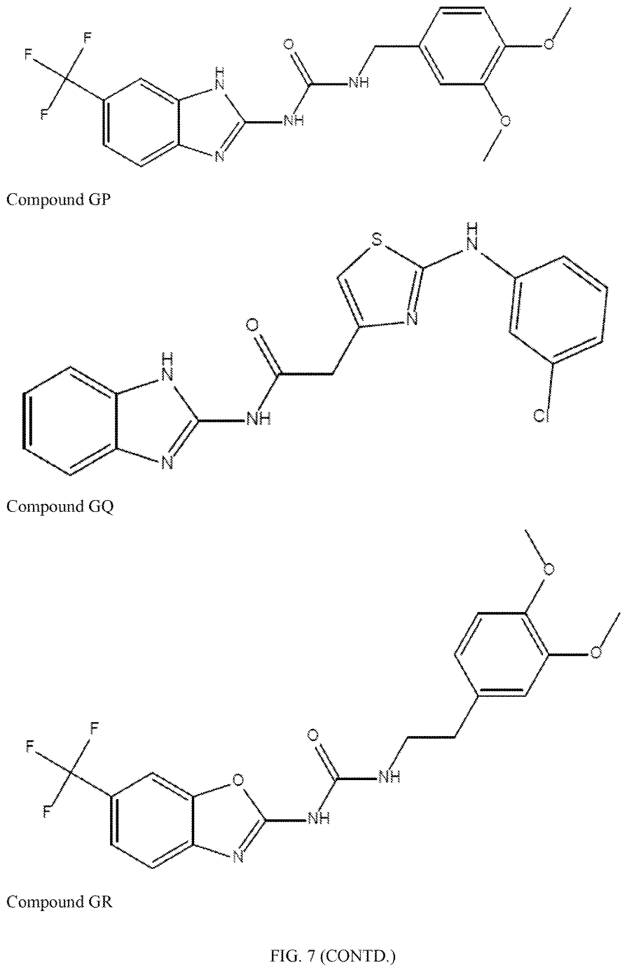

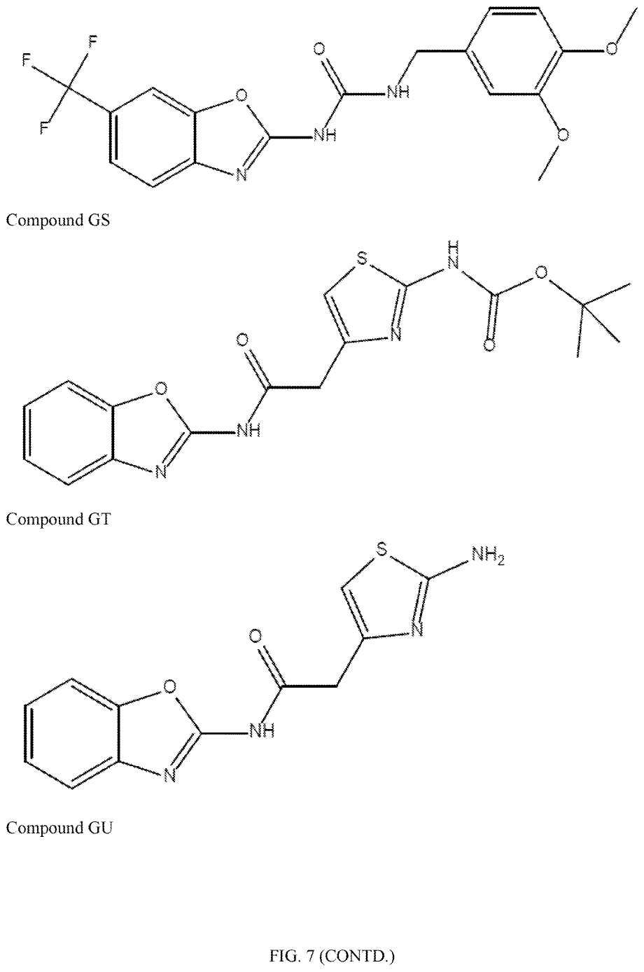

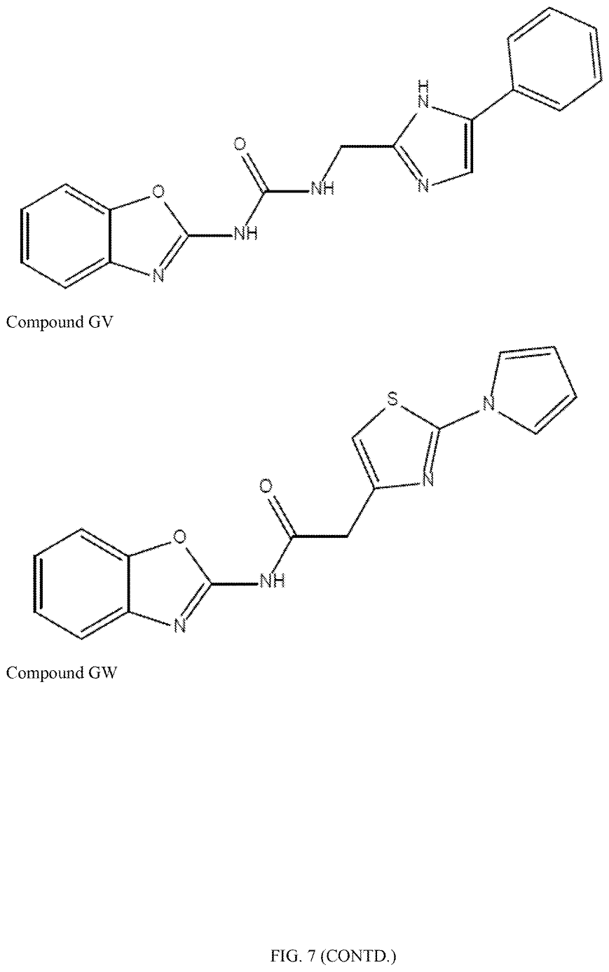

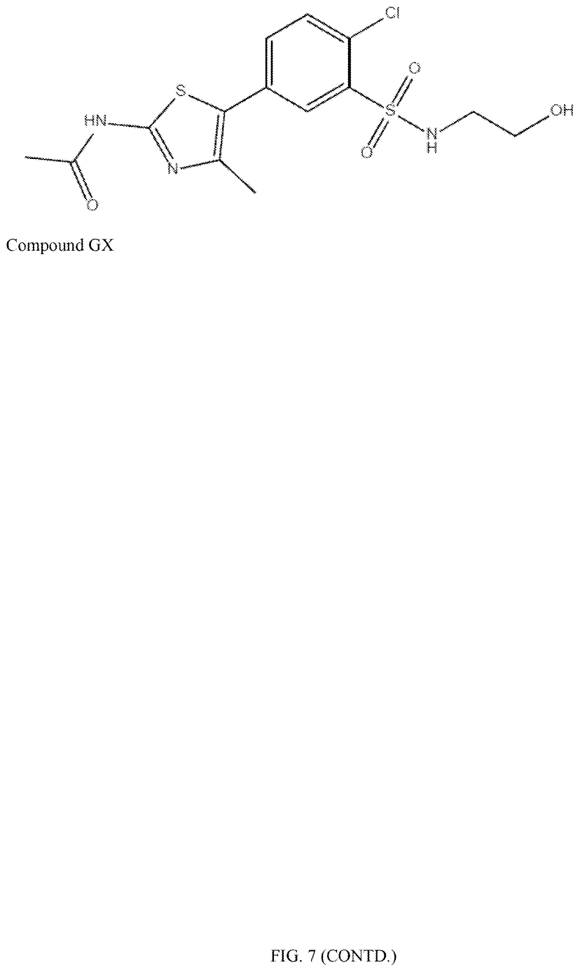

FIG. 7 is a non-limiting list of inhibitor compounds of the claimed invention. In certain embodiments, an inhibitor compound has the formula and structure of C₁₉H₁₆N₆OS₂ (Compound A); C₁₉H₁₅BrN₆OS₂ (Compound B); C₁₆H₁₄N₄OS₂ (Compound C); C₁₉H₁₅FN₆OS₂ (Compound D); C₂₀H₁₈N₆O₂S₂ (Compound E); C₂₀H₁₅F₃N₆OS₂ (Compound F); C₂₀H₁₅F₃N₆O₂S₂ (Compound G); C₂₀H₁₅N₇OS₂ (Compound H); C₁₉H₁₆N₆O₂S₂ (Compound I); C₂₁H₁₈N₆O₄S₂ (Compound J); C₁₉H₁₇N₆O₅PS₂ (Compound K); C₃₀H₃₄N₈O₃S₂ (Compound L); C₂₃H₂₀N₆O₆S₂ (Compound M); C₂₅H₂₂N₆O₈S₂ (Compound N); C₁₇H₁₄N₆OS₃ (Compound O); C₁₉H₁₅BrN₆S₃ (Compound P); (C₂₀H₁₅Br₂N₅OS₂ (Compound Q); C₂₁H₁₅BrN₆OS₃ (Compound R); C₁₇H₁₄N₆OS₂ (Compound S); C₁₈H₁₅N₇OS₂ (Compound T); C₁₈H₁₄N₆O₂S₂ (Compound U); C₁₈H₁₄BrN₇OS₂ (Compound V); C₂₃H₁₇BrN₆OS₂ (Compound W); C₁₉H₁₄BrClN₆OS₂ (Compound X); C₂₀H₁₄BrF₃N₆OS₂ (Compound Y); C₂₀H₁₄BrF₃N₆OS₂ (Compound Z); C₂₀H₁₆BrN₅OS₂ (Compound AA); C₂₁H₁₈BrN₅O₂S₂ (Compound AB); C₂₀H₁₅BrClN₅OS₂ (Compound AC); C₁₈H₁₄N₄O₃S₂ (Compound AD); C₁₆H₁₂N₄OS₂ (Compound AE); C₁₅H₁₄N₄O₃S₂ (Compound AF); C₁₇H₁₆N₄O₄S₂ (Compound AG); C₁₈H₁₈N₄O₃S₂ (Compound AH); C₁₇H₁₆N₂O₅S₃ (Compound AI); C₂₀H₁₃ClN₂O₅S₂ (Compound AJ); C₁₇H₁₃N₃O₃S (Compound AK); C₁₄H₁₀N₆OS₂ (Compound AL); C₁₈H₁₃N₃O₅S₂ (Compound AM); C₁₈H₁₆N₂O₅S₂ (Compound AN); C₂₀H₂₃N₅O₃S (Compound AO); C₁₈H₁₈N₄O₄S₂ (Compound AP); C₁₆H₁₃ClN₂O₃S₃ (Compound AQ); C₁₇H₁₄N₄O₃S₃ (Compound AR); C₁₈H₁₆N₆O₃S (Compound AS); C₁₉H₁₅ClN₄O₂S (Compound AT); C₁₇H₁₂ClN₃O₂S₃ (Compound AU); C₁₆H₁₄N₄O₄S₂ (Compound AV); C₁₈H₂₀N₄O₃S₂ (Compound AW); C₁₄H₁₄N₄O₂S₃ (Compound AX); C₁₆H₁₄N₄O₄S₂ (Compound AY); C₂₁H₁₆N₄O₅S₂ (Compound AZ); C₁₈H₁₃N₃O₄S₂ (Compound BA); C₁₃H₁₂N₄O₃S₃ (Compound BB); C₂₂H₁₉N₃O₄S₂ (Compound BC); C₁₇H₁₄N₄O₃S₂ (Compound BD); C₁₇H₁₆N₂O₃S₂ (Compound BE); C₁₇H₁₆N₂O₃S₃ (Compound BF); C₂₀H₁₅ClN₄O₄S₃ (Compound BG); C₁₆H₁₂N₄OS₂ (Compound BH); C₁₆H₁₂N₄O₃S₃ (Compound BI); C₁₇H₁₄N₆O₃S₃ (Compound BJ); C₁₈H₁₆N₆O₄S₂ (Compound BK); C₁₇H₁₃BrN₆O₃S₂ (Compound BL); C₁₇H₁₃ClN₆O₃S₂ (Compound BM); C₁₇H₁₄N₆O₃S₂ (Compound BN); C₁₈H₁₅N₅O₃S₂ (Compound BO); C₁₈H₁₅N₅O₃S₂ (Compound BP); C₁₈H₁₄BrN₅OS (Compound BQ); C₁₈H₁₃BrFN₅OS (Compound BR); C₁₈H₁₃Br₂N₅OS (Compound BS); C₁₉H₁₃BrF₃N₅OS (Compound BT); C₁₈H₁₂BrF₂N₅OS (Compound BU); C₁₈H₁₄N₄O₂S (Compound BV); C₁₉H₁₆BrN₅O₃S₂ (Compound BW); C₁₈H₁₃BrClN₅OS (Compound BX); C₂₀H₁₈BrN₅O₂S (Compound BY); C₁₈H₂₃BrN₄OS (Compound BZ); C₁₉H₁₃BrF₃N₅O₂S (Compound CA); C₁₉H₁₃BrF₃N₅OS (Compound CB); C₁₉H₁₃BrF₃N₅OS (Compound CC); C₁₉H₁₃BrF₃N₅OS (Compound CD); C₁₄H₁₀F₃N₃O₃S₃ (Compound CE); C₁₇H₁₃N₃O₄S₃ (Compound CF); C₁₇H₁₃N₃O₃S₄ (Compound CG); C₁₈H₁₉N₅O₂S₂ (Compound CH); C₁₉H₁₅N₅O₂S₂ (Compound CI); C₂₁H₁₅ClN₄O₂S₂ (Compound CJ); C₁₄H₁₂N₄O₂S₂ (Compound CK); C₁₉H₁₆N₄OS₂ (Compound CL); C₁₈H₁₆N₆OS₂ (Compound CM); C₁₉H₁₃ClN₄O₂S₂ (Compound CN); C₁₇H₁₂N₆O₂S₂ (Compound CO); C₁₇H₁₂N₆O₂S₂ (Compound CP); C₁₈H₁₄N₄OS₂ (Compound CQ); C₁₉H₁₃ClN₄O₂S₂ (Compound CR); C₁₇H₁₂N₄O₃S₂ (Compound CS); C₁₉H₁₄ClN₅O₂S₂ (Compound CT); C₂₀H₁₆N₄O₃S₂ (Compound CU); C₁₈H₁₉N₃O₃S₂ (Compound CV); C₁₈H₁₈N₂O₅S₂ (Compound CW); C₂₀H₂₁ClN₄O₂S (Compound CX); C₁₈H₁₆F₃N₃O₃S (Compound CY); C₁₈H₁₉N₃O₅S₂ (Compound CZ; C₂₀H₂₁FN₄O₂S (Compound DA); C₂₀H₂₂N₄O₂S (Compound DB); C₁₈H₁₄ClN₅OS₂ (Compound DC); C₂₀H₁₅F₃N₄OS₂ (Compound DD); C₂₀H₁₈N₄O₃S₂ (Compound DE); C₂₀H₁₇FN₄O₂S₂ (Compound DF); C₂₀H₁₅ClN₄O₂S₂ (Compound DG); C₂₀H₁₇ClN₄O₂S₂ (Compound DH); C₂₁H₁₇F₃N₄O₂S₂ (Compound DI); C₂₀H₁₆ClN₅O₂S₂ (Compound DJ); C₂₀H₁₈N₄O₂S₂ (Compound DK); C₂₀H₁₅F₃N₄O₂S₂ (Compound DL); C₁₉H₁₅N₃OS₂ (Compound DM); C₂₀H₁₇N₃OS₂ (Compound DN); C₂₀H₁₇N₃OS₂ (Compound DO); C₂₁H₁₉N₃O₂S₂ (Compound DP); C₂₀H₁₇N₃OS₂ (Compound DQ); C₂₀H₁₇N₃O₂S₂ (Compound DR); C₂₂H₂₁N₃OS₂ (Compound DS); C₂₁H₁₉N₃OS₂ (Compound DT); C₂₂H₁₆F₃N₃O₂S (Compound DU); C₂₂H₁₆F₃N₃OS (Compound DV); C₁₉H₁₈F₃N₃O₃S (Compound DW); C₂₂H₁₉N₃O₄S₂ (Compound DX); C₂₂H₁₉N₃O₃S₂ (Compound DY); C₁₉H₁₆N₄O₃S₃ (Compound DZ); C₂₁H₁₈ClN₅O₃S₂ (Compound EA); C₂₀H₁₆ClN₅O₂S₂ (Compound EB); C₁₉H₁₄ClN₅O₂S₂ (Compound EC); C₂₀H₁₆ClN₅O₂S₂ (Compound ED); C₁₉H₂₁N₃O₅S₂ (Compound EE); C₁₉H₁₈F₃N₃O₄S (Compound EF); C₁₈H₁₆F₃N₃O₄S (Compound EG); C₂₂H₁₆F₃N₃OS (Compound EH); C₂₂H₁₆F₃N₃O₂S (Compound EI); C₂₃H₁₈F₃N₃O₂S (Compound EJ); C₂₂H₁₉N₃O₄S₂ (Compound EK); C₁₇H₁₈N₄O₃S₂ (Compound EL); C₁₂H₁₀N₄OS₂ (Compound EM); C₁₉H₁₃FN₄O₂S₂ (Compound EN); C₁₈H₂₀N₄O₅S₃ (Compound EO); C₁₃H₁₂N₄O₃S₃ (Compound EP); C₁₉H₂₀N₄O₅S₃ (Compound EQ); C₁₇H₁₄F₃N₃O₄S₂ (Compound ER); C₁₆H₁₄ClN₃O₃S₂(Compound ES); C₁₄H₁₄N₄O₃S₃ (Compound ET); C₁₉H₁₇N₅O₃S₂ (Compound EU); C₁₆H₁₄BrN₃O₃S₂ (Compound EV); C₁₈H₁₅N₃O₃S₂ (Compound EW); C₁₉H₂₁N₅O₄S₃ (Compound EX); C₂₀H₁₆N₄O₄S₃ (Compound EY); C₁₉H₁₆N₄O₃S₃ (Compound EZ); C₁₈H₁₆N₄O₃S₂ (Compound FA); C₂₀H₂₃N₃O₃S₂ (Compound FB); C₁₈H₁₄FN₃O₃S₂ (Compound FC); C₁₇H₁₃N₅O₄S₄ (Compound FD); C₁₇H₁₅FN₄O₄S₃ (Compound FE); C₂₁H₁₅F₃N₄O₅S₃ (Compound FF); C₁₈H₁₅N₅OS (Compound FG); C₁₈H₁₆N₄O₃S₂ (Compound FH); C₂₁H₁₈N₄O₅S₃ (Compound FI); C₂₁H₁₅F₃N₄O₄S₃ (Compound FJ); C₂₀H₁₈N₄O₃S₂ (Compound FK); C₁₇H₁₄N₆O₃S₃ (Compound FL); C₂₁H₁₈N₄O₄S₃ (Compound FM); C₁₉H₁₆N₄O₃S₃ (Compound FN); C₂₀H₁₄Cl₂N₄O₄S₃ (Compound FO); C₁₄H₁₃N₃O₃S₃ (Compound FP); C₁₇H₁₅N₅O₃S₂ (Compound FQ); C₁₈H₁₆N₄O₃S₂ (Compound FR); C₁₈H₁₅N₃O₃S₃ (Compound FS); C₂₀H₁₅ClN₄O₄S₃ (Compound FT); C₁₉H₁₆BrN₅O₃S₂ (Compound FU); C₁₉H₁₆N₄O₃S₂ (Compound FV); C₂₂H₁₉N₃O₃S₂ (Compound FW); C₁₉H₂₁N₃O₅S₂ (Compound FX); C₁₉H₁₆N₄OS₂ (Compound FY); C₂₀H₁₇ClN₄O₂S₂ (Compound FZ); C₂₀H₁₈N₄O₂S₂ (Compound GA); C₁₉H₁₅ClN₄OS₂ (Compound GB); C₁₈H₁₄ClN₅OS₂ (Compound GC); C₁₈H₁₃ClN₄OS₂ (Compound GD); C₁₉H₁₅ClN₄OS₂ (Compound GE); C₁₉H₁₆N₄OS₂ (Compound GF); C₁₈H₁₄N₄OS (Compound GH); C₁₈H₁₃BrN₄OS (Compound GI); C₁₉H₁₆N₄O₂S (Compound GJ); C₁₇H₁₉N₅O₃S (Compound GK); C₁₂H₁₁N₅OS (Compound GL); C₁₉H₁₇N₅OS (Compound GM); C₁₈H₁₆N₆O (Compound GN); C₁₉H₁₉F₃N₄O₃ (Compound GO); C₁₈H₁₇F₃N₄O₃ (Compound GP); C₁₈H₁₄ClN₅OS (Compound GQ); C₁₉H₁₈F₃N₃O₄ (Compound GR); C₁₈H₁₆F₃N₃O₄ (Compound GS); C₁₇H₁₈N₄O₄S (Compound GT); C₁₂H₁₀N₄O₂S (Compound GU); C₁₈H₁₅N₅O₂ (Compound GV); C₁₆H₁₂N₄O₂S (Compound GW); or C₁₄H₁₆ClN₃O₄S₂ (Compound GX).

Certain embodiments are directed to pharmaceutical compositions comprising one or more of inhibitor compounds.

The inhibitor compounds of the claimed invention were tested at a concentration of M to determine percentage inhibition of the CdFabK activity. This information was used to identify potent compounds for IC₅₀ determinations (>50% inhibition) and MIC testing against C. difficile R20291.

Pyridine Head Group Modifications (5a-i)

Table 1 shows the biochemical and microbiological activity of the compounds synthesized with pyridine head group modifications, along with other relevant activity data. To determine the importance of the 2-pyridine moiety for inhibitory activity, the pyrimidine counterpart 5b was synthesized and tested. Compound 5b showed comparable activity to 5a, with an IC₅₀ of 1.02 M (vs. 1.23 μM) and an MIC of 1.56-3.12 g/mL (vs. 1.56 μg/mL). Interestingly, the IC₅₀ of compound 5d, with ap-bromophenyl group substituted for the pyridine group, was 2-fold greater than that of 5a (0.53 vs 1.23 μM). Notably, 5b displayed partial inhibition to ~65 percent, which influenced the calculated IC₅₀. Several of the compounds in this series showed a pattern of partial inhibition due to low aqueous solubility when tested at higher concentrations.

Compounds bearing electron-withdrawing groups (EWG) at the 5-position of the pyridine ring were also tested. Compounds 5e and 5f showed similar inhibition patterns as 5d with IC₅₀'s of 0.85 M and 0.77 μM, respectively. These compounds also showed partial inhibition, although not at the same extent as 5d. With respect to their anti-C. difficile activity, compounds 5e and 5f were one-fold less active with MIC's of 3.12 g/mL. Notably, 5g with a 3-CF₃ substitution showed significantly improved FabK inhibition (IC₅₀ 0.24 M) and anti-C. difficile activity equipotent to 5a with MIC of 1.56 μg/mL. Larger bicyclic substitutions of the pyridine group, quinoline 5 h and benzothiazole 5i, were largely inactive against C. difficile (MIC's>50 g/mL), although they possessed sub-micromolar IC₅₀'s of 0.75 μM and 0.42 μM, respectively. It is unclear how the bulkier ring substitutions diminished antibacterial activity but decreased cellular uptake and/or efflux pump are potential reasons. All compounds tested in this series, except 5g and 5i, showed good curve fit statistics (R²=0.95-0.97) and Hill slope values near unity, indicating ideal stoichiometric inhibitor behavior (one inhibitor to one active site). The Hill slopes significantly less than unity of 5g and 5i indicate non-ideal behavior and may be indicative of solubility or compound aggregation issues.

TABLE 1
CdFabK Inhibition and Antibacterial Activity of Pyridine-Headed Modifications

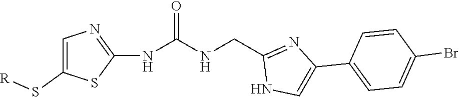

| | | R-S S | HN | maximum | | | R20291 |
|---|---|---|---|---|---|---|---|
| | | % inhibition | IC 50 | inhibition | | | MIC |
| Compound | R-group | at 10 μM | (μM) | (%) | Hill slope | R² | (μg/mL) |
| 5a | 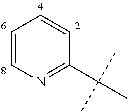 | 85.5 | 1.23 | 93 | 1.07 | 0.95 | 1.56 |
| 5b | 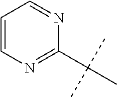 | 78.1 | 1.02 | 92 | 0.82 | 0.97 | 1.56-3.12 |
| 5c | 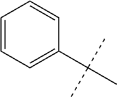 | 84.7 | 0.90 | 91 | 0.95 | 0.93 | 1.56-3.12 |
| 5d | 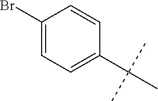 | — | 0.53 | 65 | 0.83 | 0.95 | 25 |
| 5e | 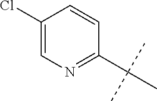 | 70.3 | 0.85 | 89 | 1.39 | 0.98 | 3.12 |
| 5f | 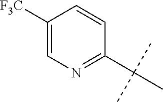 | 69.7 | 0.77 | 89 | 1.06 | 0.97 | 3.12 |
| 5g | 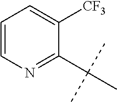 | 85.5 | 0.24 | 100 | 0.66 | 0.98 | 1.56 |
| 5h | 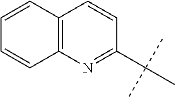 | 68.7 | 0.75 | 77 | 1.16 | 0.95 | >50 |
| 5i | 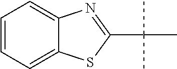 | — | 0.42 | 76 | 0.69 | 0.98 | >50 |
| Vancomycin | | | | | | | 0.39-0.78 |
| Cerulenin | | | | | | | 3.12 |

Benzothiazole Derivatives (6a-l) and Central Planar Group Modifications (7a-bb)

Compounds that replaced the 2-pyridine thioether head group with a benzothiazole ring system were tested. This substitution affected both the head group and central planar regions. Table 2 shows the biochemical and microbiological activity of the compounds in this series. Except for 6k, the curve fit statistics and Hill slopes calculated were within the acceptable range for a competitive enzyme inhibitor. Compound 6a, with an unsubstituted benzothiazole system, had a calculated IC₅₀ of 1.88 μM, but was inactive against C. difficile in MIC testing. Improvements in activity were seen by the addition of electron withdrawing functional groups, including halogen substitutions, CF₃, and methyl sulfone. Compound 6d, bearing a 6-CF₃ substitution showed excellent CdFabK inhibition (IC₅₀=0.10 μM), but an MIC of 6.25 μg/mL. Slight partial inhibition was observed for this compound. Compound 6f, bearing a stronger EWG methyl sulfone, showed slightly less enzyme inhibition (IC₅₀=0.45 μM), however it was inactive against C. difficile (MIC>100 μg/mL) presumably due to decreased cellular penetration. Compounds 6g, 6h, and 6i (6-Br, 6-Cl, and 6-F, respectively) showed CdFabK inhibition with IC₅₀'s ranging from 0.11 to 0.34 μM. Interestingly, 6 h showed the most potent anti-C. difficile activity with an MIC of 6.25 μg/mL. In contrast, compound 6j, possessing a 4,6-difluoro substitution, showed significantly less enzyme inhibition (IC₅₀=4.53 μM) and anti-C. difficile activity (MIC=12.5 μg/mL). To further explore the SAR of this series, the effect of electron donating groups on biochemical and microbiological activity was investigated. Compound 6k, bearing a 6-ethoxy group, showed promising CdFabK inhibition (IC₅₀=0.17 μM) with anti-C. difficile activity of MIC=12.5 μg/mL. The low Hill slope of this compound suggests a non-ideal inhibitory behavior, possibly due to poor solubility or aggregation. Compound 6l, bearing a trifluoromethoxy group, showed excellent activity in the nanomolar range (IC₅₀=0.09 μM) with moderate anti-C. difficile activity (MIC 12.5-25 μg/mL).

Modifications of the urea moiety in the central planar region of 5a were explored by substitution of the urea with a thiourea, giving 7a. Compound 7a showed CdFabK inhibitory activity that was comparable to 5a (IC₅₀=1.77 μM), but significantly improved anti-C. difficile activity with an MIC of 0.39 μg/mL, which is comparable to vancomycin. However, the thiourea functionality is a well-known toxicophore, necessitating caution in future investigations of this scaffold. In contrast, removal of the central planar system in compound 7b abolished enzymatic and cellular inhibition (IC₅₀>100 μM, MIC>50 μg/mL), confirming that a planar group is essential.

TABLE 2
CdFabK Inhibition and Antibacterial Activity of 6a-1 with substituted
Benzothiazole Rings

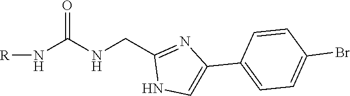

| | | | | maximum | | | R20291 |
|---|---|---|---|---|---|---|---|
| | | % inhibition | IC₅₀ | inhibition | Hill | | MIC |
| Compound | R group | at 10 μM | (μM) | (%) | slope | R² | (μg/mL) |
| 6a | 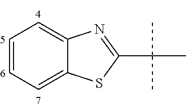 | 78.3 | 1.88 | 95 | 0.94 | 0.98 | >50 |
| 6b | 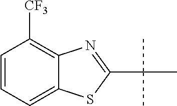 | 10.2 | >10 | — | — | — | 1.56 |
| 6c | 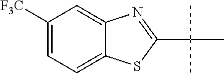 | 41.0 | >10 | — | — | — | 3.12 |
| 6d | 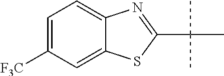 | 88.5ª | 0.10a | 89 | 1.07 | 0.99 | 6.25 |
| 6e | 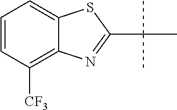 | 65.1 | 2.81 | 71 | 1.01 | 0.98 | 12.5 |
| 6f | 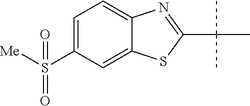 | 91.3 | 0.45 | 95 | 0.97 | 0.98 | >100 |
| 6g | 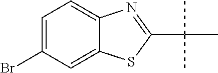 | 93.6ª | 0.34 | 99 | 1.02 | 0.94 | 50 |
| 6h | 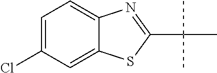 | 88.9 | 0.11 | 96 | 0.86 | 0.99 | 6.25 |
| 6i | 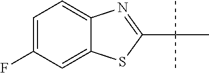 | 93.1a | 0.23 | 98 | 0.85 | 0.96 | ND^{b} |
| 6j | 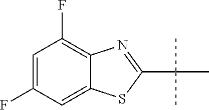 | 69.9 | 4.53 | 79 | 1.18 | 0.97 | 12.5 |
| 6k | 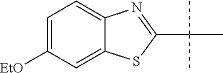 | 73.8 | 0.17 | 91 | 0.67 | 0.97 | 12.5 |
| 61 | 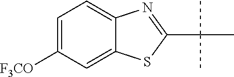 | 86.3 | 0.09 | 91 | 0.84 | 0.95 | 12.5-25 |
| Vancomycin | | | | | | | 0.39-0.78 |
| Cerulenin | | | | | | | 3.12 |

^{a}The protein concentration used in these assays was 4 nM.
^{b}Not determined.

Phenylimidazole Tail Group Modifications (8a-c)

In the modification of the phenylimidazole tail group, compounds 8a-c were synthesized to explore tail group substitutions of phenyl-triazole (8a), benzimidazole (8b), and quinazolinone (8c) as shown in Table 3. These compounds were designed to retain the recognized hydrogen bond donor and acceptor groups of the phenylimidazole discussed above, while exploring differing bicyclic ring systems and steric bulk. As shown in Table 3, overall these compounds showed low inhibitory activity against CdFabK (IC₅₀>10 μM) and no anti-C. difficile activity with MICs of >50 μg/mL. This suggests that the tail region of this inhibitor series is significantly less tolerant to substitution compared to the head and central regions.

TABLE 3
CdFabK Inhibition and Antibacterial Activity of 8a-c with Various Tail Groups

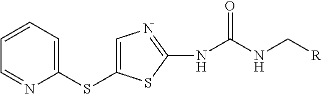

| | | % | | R20291 |
|---|---|---|---|---|
| | | inhibition | IC₅₀ (μM) | MIC |
| Compound | R | at 10 μM | CdFabK | (μg/mL) |
| 8a | 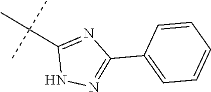 | 28.1 | >10 | >50 |
| 8b | 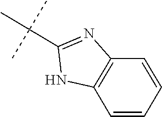 | 43.7 | >10 | >50 |
| 8c | 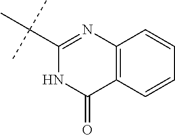 | 26.2 | >10 | >50 |

### Compound Solubility

To investigate the experimental solubility, select compounds from the 5a-i and 6a-l series were tested using a turbidimetric assay previously reported and results are shown in Table 4. The most soluble compound. 6j, with an estimated precipitation point of 125 μM, showed low inhibition activity against CdFabK (IC₅₀=4.53 μM) and pyrimidine analog 5b had a precipitation point of 83.9 μM. The nature of the endogenous FabK substrate (fatty acids) and the observed lipophilic regions of the CdFabK active site suggest that lipophilic interactions are a key driver for binding affinity. The biochemical activity and solubility assay data shown here imply that a careful balance of lipophilic and hydrophilic character is required for a compound to possess potent CdFabK inhibition. For example, the most potent analogs evaluated here, compounds 5g, 6d, 6f, and 6i, have estimated precipitation points in the range of 44.5 μM to 65.3 M. This suggests that the optimal solubility, that correlates to optimal activity, can be predicted by a compound precipitation point of approximately 55 μM. Compound 6h was an unusual outlier in these studies as it showed a decrease in absorbance above the precipitation point of 76.9 μM, indicating the compound's solubility was improving at higher concentrations. It is noted that the low Hill slope of 6h may imply aggregation or micelle formation is occurring with this compound.

TABLE 4
Solubility Assay Data of Select Compound Set

| | | Estimated | |
|---|---|---|---|
| | | precipitation point | |
| Entry | Compound | (μM) | R² |
| 1 | 5a | 80.1 | 0.99 |
| 2 | 5b | 83.9 | 0.99 |
| 3 | 5d | 63.6 | 0.97 |
| 4 | 5e | 59.0 | 0.99 |
| 5 | 5f | 18.3 | 0.98 |
| 6 | 5g | 52.0 | 0.98 |
| 7 | 5h | 74.7 | 0.99 |
| 8 | 5i | 50.0 | 0.92 |
| 9 | 6a | 64.8 | 0.97 |
| 10 | 6d | 65.3 | 0.99 |
| 11 | 6f | 44.5 | 0.97 |
| 12 | 6g | 31.3 | 0.93 |
| 13 | 6h | 76.9^{a} | 0.98 |
| 14 | 6i | 61.3 | 0.92 |
| 15 | 6j | 125 | 0.98 |
| 16 | 6k | 79.0 | 0.69 |

^{a}Compound showed a decrease in absorbance after this point.

### WORKING EXAMPLES

The following experiments are intended to further elaborate and expand on the detailed description and are in no way intended to limit the scope of the invention, nor should they be construed as limiting the scope of the invention.

### General Methods

### Solvents and Reagents

Commercial reagents were purchased from Sigma-Aldrich or Fisher Scientific and used without additional purification. Dichloromethane (DCM), dimethylformamide (DMF), and tetrahydrofuran (THF) were purchased in a Sure/Seal bottle (≥99.8%, anhydrous) and additionally sparged with N₂ prior to use.

### Reaction Setup, Monitoring, and Product Purification

Reactions were carried out in oven-dried glassware under a positive pressure of N₂ in anhydrous solvents using standard Schlenk techniques. Reactions run at room temperature (22-23° C.) and heating temperature (80-100° C.) controlled by an IKA temperature modulator. Reaction progress was monitored using an analytical high-performance liquid chromatography (HPLC) employing a Shimadzu LC-20A series HPLC system. Compounds were purified by flash column chromatography on silica gel using a Biotage Isolera One system on prefilled, Biotage SNAP Ultra columns and Biotage SNAP NH columns using ACS grade solvents. Organic solutions were concentrated under reduced pressure on a Heidolph temperature-controlled rotary evaporator equipped with a Brinkmann Lauda WK 1200 Circulation Chiller.

### Biological Assays

### Enzyme Expression & Purification

The CdFabK enzyme was expressed following known protocols. After harvesting the cells, they were suspended in a lysis buffer consisting of Buffer A (50 mM HEPES pH 8.0, 300 mM NH₄Cl, 18% glycerol, 100 μM FMN, 10 mM imidazole, and 1 mM DTT), 0.5 mg/mL lysozyme, 0.5% Triton-X 100, 25 mM sucrose, 10 μg/mL DNase, and protease inhibitor. Cells were then lysed in a pulse sonifier with 8 seconds of ultrasonic pulse and 24 seconds of interval with a total pulse time of 8 min, and then the lysates were centrifuged at 18000 RPM for 15 minutes using a JA-20 rotor in a Beckman Coulter Centrifuge. The supernatant was passed through 0.22 μM filters. Protein purification was done by affinity chromatography in a His-Trap HP column (Cytiva Life Sciences) and eluted with an elution buffer (50 mM HEPES pH 8.0, 300 mM NH₄Cl, 18% glycerol, 100 μM FMN, 500 mM imidazole and 1 mM DTT). The protein was further purified by gel filtration on a SuperDex 200 PG column (GE Healthcare) with a buffer containing, 50 mM Hepes pH 8.0, 300 mM NH₄Cl, 18% glycerol, 100 μM FMN, and 3 mM DTT. After purification, the protein was concentrated using a centrifugal filter (Amicon Ultra-15).

### General Assay Method

The CdFabK enzyme assays were conducted at 25° C. using assay buffer (100 mM HEPES pH 8.0, 500 mM NH₄Cl, 10% glycerol) in 384-well microplates at a final volume of 100 μL (30 μL of buffer, 10 μL inhibitor, 5 μL of crotonyl-CoA substrate, 5 μL of protein, and 50 μL NADH). The final concentrations of enzyme, NADH cofactor, and crotonyl-CoA substrate were 15 nM, 125 μM, and 325 μM, respectively. Test compounds were serially diluted using DMSO prior to assay. The initial concentration of the test compounds and the dilution factor were determined by initial single concentration assays at 10 μM and 100 μM to give an appropriate concentration range with data points above and below the inflection for best curve fits. A working dilution of the protein was prepared using the assay buffer with 2.5 mg/ml γ-globulins (used for a crowding effect to stabilize the CdFabK dimer at low concentrations). The compound and enzyme were incubated for 10 minutes, after which crotonyl-CoA substrate was added. The reaction is started by the addition of the NADH cofactor and continuously monitored for NADH conversion to NAD⁺ by fluorescence intensity (ex 340, em 460) in a Biotek Synergy H1 microplate reader for 10 min total duration with reads every 20 seconds. For each test compound, eleven concentrations were tested, in duplicate, along with a DMSO control.

### IC₅₀ Determination

Slopes obtained from the steady-state period of the continuous assays (linear range) were calculated and used with control data to determine percent inhibition at each concentration. Percent inhibitions (duplicate) and compound concentrations were used to calculate IC₅₀ values for each compound by four-parameter non-linear regression (Hill equation) fit with GraphPad Prism 9.0. For a better estimate of the goodness of fit, Sy.x (Standard deviation of the Residual) was calculated with GraphPad Prism 9.0.

### Solubility Assays

Solubility of the compounds was measured using standard turbidimetric protocols. 1 mL of the CdFabK assay buffer was placed in a cuvette. From a 10 mM DMSO stock, 1 μL of test compound was added to the buffer every minute, and absorbance was measured at 620 nm. A total of 14 concentrations were tested. The precipitation point was calculated using a segmental linear regression fit to the absorbance at Y-axis and the concentration at the X-axis. The intersection of the two-line is the inflection point and is the estimated concentration where the compound begins to precipitate.

### MIC Determination

MICs were performed by the broth microdilution method, as previously described, using C. difficile strain R20291, in brain heart infusion broth (BHI, from BD Diagnostics). Compound stock solutions were prepared to 10 mg/mL in DMSO (Alfa Aesar). BHI was deoxygenated overnight in a PLAS Labs 857-OTA anaerobic chamber. Strain R20291 was grown anaerobically overnight in BHI broth in a Don Whitley A35 anaerobic at 37° C. Compounds were serially two-fold diluted (100-0.1 μg/mL) and incubated anaerobically for 1 h before being inoculated with 100 μL of bacteria prepared as a 1:100 dilution of the overnight culture. After 24 h of anaerobic incubation at 37° C., MICs were recorded as the lowest concentration of compound that inhibited visible growth. MICs reported were performed in biological duplicates and values are shown as a range, where found.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

Conditional language used herein, such as, among others, “can”, “might”. “may”, “e.g.”, and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, the processes described herein can be embodied within a form that does not provide all of the features and benefits set forth herein, as some features can be used or practiced separately from others. The scope of protection is defined by the appended claims rather than by the foregoing description. All changes which come within the meaning and range of equivalency of the claims are to be embraced within their scope.

Although various embodiments of the method and apparatus of the present invention have been illustrated in the accompanying Drawings and described in the foregoing Detailed Description, it will be understood that the invention is not limited to the embodiments disclosed, but is capable of numerous rearrangements, modifications and substitutions without departing from the spirit of the invention as set forth herein.

What is claimed is:

1. A composition comprising an inhibitor of FabK activity.

2. The composition of claim 1, wherein the inhibitor is a compound having a structure selected from the group consisting of an imidazole scaffold, a thiazole scaffold, a pyridine-thiazole scaffold, a benzothiazole scaffold, a benzimidazole scaffold, a benzoxazole scaffold and combinations thereof.

3. The composition of claim 1, wherein the inhibitor is a phenylimidazole compound.

4. The composition of claim 3, wherein the phenylimidazole compound has a substitution to the pyridine head group.

5. The composition of claim 3, wherein the phenylimidazole compound has a modification to the pyridine head group.

6. The composition of claim 3, wherein the head group of the phenylimidazole compound is modified by the addition of one or more halogen groups.

7. The composition of claim 3 wherein, the phenylimidazole compound comprises modifications to the planar central aromatic region.

8. The composition of claim 7, wherein a thiazole of the central aromatic region is substituted with quinoline and/or benzothiazole.

9. The composition of claim 7, wherein the urea is replaced with a thiourea.

10. The composition of claim 3, wherein the phenylimidazole compound comprises modifications to the aromatic tail group ring.

11. The composition of claim 10, wherein the modifications to the aromatic tail group involves substitution of the phenylimidazole group with aromatic bicyclic groups.

12. A compound having the structure: 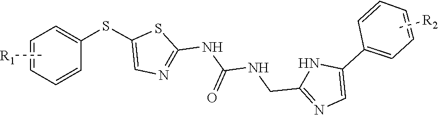
wherein:
R₁ and R₂ are a halogen, —OCH₃, —CF₃, —CN or —OCF₃.

13. A compound having the structure: 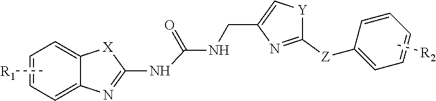
wherein:
X is S, N or O;
R₁ is —CF₃, —CH₃, —SO₂CH₃, halogen, —OCH₃, —OCOCH₃, —OC₂H₅ or —SO₂N(CH₃)₂;
Z, when present, is NH, NHCO or NHCONH;
Y is S or N; and
R₂ is a halogen, —OCH₃, —CF₃, —CN or —OCF₃.

14. A compound having the structure: 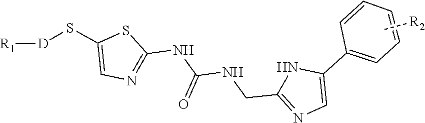
wherein:
D is a benzene, pyridine, pyrimidine, benzothiazole, or quinoline; and
R₁ and R₂ are a halogen, —OCH₃, —CF₃, —CN or —OCF₃.

15. A compound selected from the group consisting of:
C₁₉H₁₆N₆OS₂ (Compound A); C₁₉H₁₅BrN₆OS₂ (Compound B); C₁₆H₁₄N₄OS₂ (Compound C); C₁₉H₁₅FN₆OS₂ (Compound D); C₂₀H₁₈N₆O₂S₂ (Compound E); C₂₀H₁₅F₃N₆OS₂ (Compound F); C₂₀H₁₅F₃N₆O₂S₂ (Compound G); C₂₀H₁₅N₇OS₂ (Compound H); C₁₉H₁₆N₆O₂S₂ (Compound I); C₂₁H₁₈N₆O₄S₂ (Compound J); C₁₉H₁₇N₆O₅PS₂ (Compound K); C₃₀H₃₄N₈O₃S₂ (Compound L); C₂₃H₂₀N₆O₆S₂ (Compound M); C₂₅H₂₂N₆O₈S₂ (Compound N); C₁₇H₁₄N₆OS₃ (Compound O); C₁₉H₁₅BrN₆S₃ (Compound P); (C₂₀H₁₅Br₂N₅OS₂ (Compound Q); C₂₁H₁₅BrN₆OS₃ (Compound R); C₁₇H₁₄N₆OS₂ (Compound S); C₁₈H₁₅N₇OS₂ (Compound T); C₁₈H₁₄N₆O₂S₂ (Compound U); C₁₈H₁₄BrN₇OS₂ (Compound V); C₂₃H₁₇BrN₆OS₂ (Compound W); C₁₉H₁₄BrClN₆OS₂ (Compound X); C₂₀H₁₄BrF₃N₆OS₂ (Compound Y); C₂₀H₁₄BrF₃N₆OS₂ (Compound Z); C₂₀H₁₆BrN₅OS₂ (Compound AA); C₂₁H₁₈BrN₅O₂S₂ (Compound AB); C₂₀H₁₅BrClN₅OS₂ (Compound AC); C₁₈H₁₄N₄O₃S₂ (Compound AD); C₁₆H₁₂N₄OS₂ (Compound AE); C₁₅H₁₄N₄O₃S₂ (Compound AF); C₁₇H₁₆N₄O₄S₂ (Compound AG); C₁₈H₁₈N₄O₃S₂ (Compound AH); C₁₇H₁₆N₂O₅S₃ (Compound AI); C₂₀H₁₃ClN₂O₅S₂ (Compound AJ); C₁₇H₁₃N₃O₃S (Compound AK); C₁₄H₁₀N₆OS₂ (Compound AL); C₁₈H₁₃N₃O₅S₂ (Compound AM); C₁₈H₁₆N₂O₅S₂ (Compound AN); C₂₀H₂₃N₅O₃S (Compound AO); C₁₈H₁₈N₄O₄S₂ (Compound AP); C₁₆H₁₃ClN₂O₃S₃ (Compound AQ); C₁₇H₁₄N₄O₃S₃ (Compound AR); C₁₈H₁₆N₆O₃S (Compound AS); C₁₉H₁₅ClN₄O₂S (Compound AT); C₁₇H₁₂ClN₃O₂S₃ (Compound AU); C₁₆H₁₄N₄O₄S₂ (Compound AV); C₁₈H₂₀N₄O₃S₂ (Compound AW); C₁₄H₁₄N₄O₂S₃ (Compound AX); C₁₆H₁₄N₄O₄S₂ (Compound AY); C₂₁H₁₆N₄O₅S₂ (Compound AZ); C₁₈H₁₃N₃O₄S₂ (Compound BA); C₁₃H₁₂N₄O₃S₃ (Compound BB); C₂₂H₁₉N₃O₄S₂ (Compound BC); C₁₇H₁₄N₄O₃S₂ (Compound BD); C₁₇H₁₆N₂O₃S₂ (Compound BE); C₁₇H₁₆N₂O₃S₃ (Compound BF); C₂₀H₁₅ClN₄O₄S₃ (Compound BG); C₁₆H₁₂N₄OS₂ (Compound BH); C₁₆H₁₂N₄O₃S₃ (Compound BI); C₁₇H₁₄N₆O₃S₃ (Compound BJ); C₁₈H₁₆N₆O₄S₂ (Compound BK); C₁₇H₁₃BrN₆O₃S₂ (Compound BL); C₁₇H₁₃ClN₆O₃S₂ (Compound BM); C₁₇H₁₄N₆O₃S₂ (Compound BN); C₁₈H₁₅N₅O₃S₂ (Compound BO); C₁₈H₁₅N₅O₃S₂ (Compound BP); C₁₈H₁₄BrN₅OS (Compound BQ); C₁₈H₁₃BrFN₅OS (Compound BR); C₁₈H₁₃Br₂N₅OS (Compound BS); C₁₉H₁₃BrF₃N₅OS (Compound BT); C₁₈H₁₂BrF₂N₅OS (Compound BU); C₁₈H₁₄N₄O₂S (Compound BV); C₁₉H₁₆BrN₅O₃S₂ (Compound BW); C₁₈H₁₃BrClN₅OS (Compound BX); C₂₀H₁₈BrN₅O₂S (Compound BY); C₁₈H₂₃BrN₄OS (Compound BZ); C₁₉H₁₃BrF₃N₅O₂S (Compound CA); C₁₉H₁₃BrF₃N₅OS (Compound CB); C₁₉H₁₃BrF₃N₅OS (Compound CC); C₁₉H₁₃BrF₃N₅OS (Compound CD); C₁₄H₁₀F₃N₃O₃S₃ (Compound CE); C₁₇H₁₃N₃O₄S₃ (Compound CF); C₁₇H₁₃N₃O₃S₄ (Compound CG); C₁₈H₁₉N₅O₂S₂ (Compound CH); C₁₉H₁₅N₅O₂S₂ (Compound CI); C₂₁H₁₅ClN₄O₂S₂ (Compound CJ); C₁₄H₁₂N₄O₂S₂ (Compound CK); C₁₉H₁₆N₄OS₂ (Compound CL); C₁₈H₁₆N₆OS₂ (Compound CM); C₁₉H₁₃ClN₄O₂S₂ (Compound CN); C₁₇H₁₂N₆O₂S₂ (Compound CO); C₁₇H₁₂N₆O₂S₂ (Compound CP); C₁₈H₁₄N₄OS₂ (Compound CQ); C₁₉H₁₃ClN₄O₂S₂ (Compound CR); C₁₇H₁₂N₄O₃S₂ (Compound CS); C₁₉H₁₄ClN₅O₂S₂ (Compound CT); C₂₀H₁₆N₄O₃S₂ (Compound CU); C₁₈H₁₉N₃O₃S₂ (Compound CV); C₁₈H₁₈N₂O₅S₂ (Compound CW); C₂₀H₂₁ClN₄O₂S (Compound CX); C₁₈H₁₆F₃N₃O₃S (Compound CY); C₁₈H₁₉N₃O₅S₂ (Compound CZ; C₂₀H₂₁FN₄O₂S (Compound DA); C₂₀H₂₂N₄O₂S (Compound DB); C₁₈H₁₄ClN₅OS₂ (Compound DC); C₂₀H₁₅F₃N₄OS₂ (Compound DD); C₂₀H₁₈N₄O₃S₂ (Compound DE); C₂₀H₁₇FN₄O₂S₂ (Compound DF); C₂₀H₁₅ClN₄O₂S₂ (Compound DG); C₂₀H₁₇ClN₄O₂S₂ (Compound DH); C₂₁H₁₇F₃N₄O₂S₂ (Compound DI); C₂₀H₁₆ClN₅O₂S₂ (Compound DJ); C₂₀H₁₈N₄O₂S₂ (Compound DK); C₂₀H₁₅F₃N₄O₂S₂ (Compound DL); C₁₉H₁₅N₃OS₂ (Compound DM); C₂₀H₁₇N₃OS₂ (Compound DN); C₂₀H₁₇N₃OS₂ (Compound DO); C₂₁H₁₉N₃O₂S₂ (Compound DP); C₂₀H₁₇N₃OS₂ (Compound DQ); C₂₀H₁₇N₃O₂S₂ (Compound DR); C₂₂H₂₁N₃OS₂ (Compound DS); C₂₁H₁₉N₃OS₂ (Compound DT); C₂₂H₁₆F₃N₃O₂S (Compound DU); C₂₂H₁₆F₃N₃OS (Compound DV); C₁₉H₁₈F₃N₃O₃S (Compound DW); C₂₂H₁₉N₃O₄S₂ (Compound DX); C₂₂H₁₉N₃O₃S₂ (Compound DY); C₁₉H₁₆N₄O₃S₃ (Compound DZ); C₂₁H₁₈ClN₅O₃S₂ (Compound EA); C₂₀H₁₆ClN₅O₂S₂ (Compound EB); C₁₉H₁₄ClN₅O₂S₂ (Compound EC); C₂₀H₁₆ClN₅O₂S₂ (Compound ED); C₁₉H₂₁N₃O₅S₂ (Compound EE); C₁₉H₁₈F₃N₃O₄S (Compound EF); C₁₈H₁₆F₃N₃O₄S (Compound EG); C₂₂H₁₆F₃N₃OS (Compound EH); C₂₂H₁₆F₃N₃O₂S (Compound EL); C₂₃H₁₈F₃N₃O₂S (Compound EJ); C₂₂H₁₉N₃O₄S₂ (Compound EK); C₁₇H₁₈N₄O₃S₂ (Compound EL); C₁₂H₁₀N₄OS₂ (Compound EM); C₁₉H₁₃FN₄O₂S₂ (Compound EN); C₁₈H₂₀N₄O₅S₃ (Compound EO); C₁₃H₁₂N₄O₃S₃ (Compound EP); C₁₉H₂₀N₄O₅S₃ (Compound EQ); C₁₇H₁₄F₃N₃O₄S₂ (Compound ER); C₁₆H₁₄ClN₃O₃S₂(Compound ES); C₁₄H₁₄N₄O₃S₃ (Compound ET); C₁₉H₁₇N₅O₃S₂ (Compound EU); C₁₆H₁₄BrN₃O₃S₂ (Compound EV); C₁₈H₁₅N₃O₃S₂ (Compound EW); C₁₉H₂₁N₅O₄S₃ (Compound EX); C₂₀H₁₆N₄O₄S₃ (Compound EY); C₁₉H₁₆N₄O₃S₃ (Compound EZ); C₁₈H₁₆N₄O₃S₂ (Compound FA); C₂₀H₂₃N₃O₃S₂ (Compound FB); C₁₈H₁₄FN₃O₃S₂ (Compound FC); C₁₇H₁₃N₅O₄S₄ (Compound FD); C₁₇H₁₅FN₄O₄S₃ (Compound FE); C₂₁H₁₅F₃N₄O₅S₃ (Compound FF); C₁₈H₁₅N₅OS (Compound FG); C₁₈H₁₆N₄O₃S₂ (Compound FH); C₂₁H₁₈N₄O₅S₃ (Compound FL); C₂₁H₁₅F₃N₄O₄S₃ (Compound FJ); C₂₀H₁₈N₄O₃S₂ (Compound FK); C₁₇H₁₄N₆O₃S₃ (Compound FL); C₂₁H₁₈N₄O₄S₃ (Compound FM); C₁₉H₁₆N₄O₃S₃ (Compound FN); C₂₀H₁₄Cl₂N₄O₄S₃ (Compound FO); C₁₄H₁₃N₃O₃S₃ (Compound FP); C₁₇H₁₅N₅O₃S₂ (Compound FQ); C₁₈H₁₆N₄O₃S₂ (Compound FR); C₁₈H₁₅N₃O₃S₃ (Compound FS); C₂₀H₁₅ClN₄O₄S₃ (Compound FT); C₁₉H₁₆BrN₅O₃S₂ (Compound FU); C₁₉H₁₆N₄O₃S₂ (Compound FV); C₂₂H₁₉N₃O₃S₂ (Compound FW); C₁₉H₂₁N₃O₅S₂ (Compound FX); C₁₉H₁₆N₄OS₂ (Compound FY); C₂₀H₁₇ClN₄O₂S₂ (Compound FZ); C₂₀H₁₈N₄O₂S₂ (Compound GA); C₁₉H₁₅ClN₄OS₂ (Compound GB); C₁₈H₁₄ClN₅OS₂ (Compound GC); C₁₈H₁₃ClN₄OS₂ (Compound GD); C₁₉H₁₅ClN₄OS₂ (Compound GE); C₁₉H₁₆N₄OS₂ (Compound GF); C₁₈H₁₄N₄OS (Compound GH); C₁₈H₁₃BrN₄OS (Compound GI); C₁₉H₁₆N₄O₂S (Compound GJ); C₁₇H₁₉N₅O₃S (Compound GK); C₁₂H₁₁N₅OS (Compound GL); C₁₉H₁₇N₅OS (Compound GM); C₁₈H₁₆N₆O (Compound GN); C₁₉H₁₉F₃N₄O₃ (Compound GO); C₁₈H₁₇F₃N₄O₃ (Compound GP); C₁₈H₁₄ClN₅OS (Compound GQ); C₁₉H₁₈F₃N₃O₄ (Compound GR); C₁₈H₁₆F₃N₃O₄ (Compound GS); C₁₇H₁₈N₄O₄S (Compound GT); C₁₂H₁₀N₄O₂S (Compound GU); C₁₈H₁₅N₅O₂ (Compound GV); C₁₆H₁₂N₄O₂S (Compound GW); and C₁₄H₁₆ClN₃O₄S₂ (Compound GX).

16. A pharmaceutical composition comprising one or compounds of claim 15.
